(19) United States
(12) Patent Application Publication
(10) Pub. No.: US 2026/0338785 A1
(43) Pub. Date: Oct. 29, 2026

(54) MULTIPLEX METHOD FOR MULTIPLE BIOMARKER DETECTION ON PARTICLES

(71) Applicant: Mercy Bioanalytics, Inc., Waltham, MA (US)

(72) Inventor: Toumy Guettouche, Waltham, MA (US)

(21) Appl. No.: 19/656,486

(22) Filed: Apr. 23, 2026

Related U.S. Application Data
(60) Provisional application No. 63/794,105, filed on Apr. 24, 2025.
(60) Provisional application No. 63/811,162, filed on May 23, 2025.
(60) Provisional application No. 64/008,057, filed on Mar. 17, 2026.
(60) Provisional application No. 64/044,660, filed on Apr. 20, 2026.
(60) Provisional application No. 64/044,661, filed on Apr. 20, 2026.

(51) Int. Cl. G01N 33/575; G01N 33/53; G01N 33/543

(52) U.S. Cl. CPC G01N 33/5759; G01N 33/5308; G01N 33/54306; G01N 33/57585; G01N 2333/70546; G01N 2333/71; G01N 2400/00; G01N 2405/00

(57) ABSTRACT

Methods, compositions, kits, and systems are provided for detecting a condition in a subject by capturing a particle from a biological sample using a capture agent specific for a first biomarker and detecting co-localized second, third, and fourth biomarkers on the same captured particle. The capture agent may bind a disease-associated biomarker for disease-specific capture, or a broadly expressed marker such as a tetraspanin, integrin, lectin target, lipid, or charge feature for pan-extracellular vesicle capture. Multi-marker co-localization is detected using proximity ligation, flow cytometry, or super-resolution microscopy, yielding high specificity for conditions including lung adenocarcinoma.

### TECHNICAL FIELD

The present disclosure relates to methods, kits, and systems for detecting cancer, such as lung cancer, by analyzing co-localized biomarker signatures on biological particles, including extracellular vesicles and non-vesicular particles.

In particular, the disclosure relates to methods for capturing extracellular vesicles and particles (EVs) and non-vesicular particles (NVPs) from biological samples using either disease-specific capture or pan-particle capture strategies, followed by highly specific detection based on co-localized multi-biomarker signatures present on individual particles.

### BACKGROUND

Cancer research has increasingly focused on analyzing biological particles shed by cells into circulation, such as Extracellular Vesicles and particles (EVs) (e.g., exosomes and microvesicles), and various Non-Vesicular Particles (NVPs) (e.g., exomeres, supermeres, lipoproteins, and protein/genomic aggregates). These particles can carry a diverse cargo of proteins, lipids, nucleic acids, and carbohydrates reflective of their cell of origin, including tumor cells. Analyzing biomarkers associated with these particles holds potential for non-invasive cancer detection.

However, achieving high diagnostic accuracy, particularly the specificity needed to distinguish early cancer from benign conditions or healthy states, remains a challenge when relying on the detection of single markers on these particles or bulk analysis of particle populations.

Healthy background signals arising from particles derived from normal tissues can obscure disease-associated signals, resulting in false positives and reduced assay performance.

There is a clear and unmet need for improved non-invasive diagnostic methods for cancers, e.g., lung cancer, that offer enhanced sensitivity and specificity for early detection, differential diagnosis, and treatment monitoring. Methods capable of detecting specific combinations of multiple biomarkers present simultaneously on individual circulating particles may provide a solution. The present disclosure addresses this need by providing novel methods focused on detecting the co-localization of multiple distinct biomarkers on target particles captured via a first biomarker, where capture of particles may be disease-specific or agnostic, and diagnostic specificity is achieved through downstream detection architecture.

### SUMMARY

The present disclosure provides novel methods, compositions, kits, and systems for the improved detection and diagnosis of cancer, e.g., lung adenocarcinoma (LUAD), through the analysis of particles derived from a subject's sample. Addressing the significant challenges in early and accurate cancer diagnosis, the methods of the present disclosure utilize involve identifying multi-marker signatures on individual particles.

Methods of the present disclosure involve capturing a target population of particles, which may be Extracellular Vesicles (EVs) or Non-Vesicular Particles (NVPs), using a capture agent specific for a first biomarker associated with a condition (e.g., a LUAD-associated protein such as EGFR or CEACAM5). Following isolation, the captured particles are analyzed to detect the simultaneous presence (i.e., co-localization) of at least three additional distinct biomarkers on the same individual particle. This requirement for detecting a complex signature involving at least four total biomarkers (one capture and at least three, four, five, or six detected) aims to confer high specificity to the assay.

In other embodiments, the methods involve capturing a broad population of particles using pan-particle or pan-extracellular vesicle (pan-EV) capture strategies that bind biomarkers broadly expressed across EVs or particles, independent of disease state or consistent with a number of disease states. In such embodiments, the capture step primarily serves to enrich particles from the sample, while diagnostic specificity is conferred primarily or entirely by the detection of co-localized biomarker combinations on individual particles.

The co-localized biomarkers may include various molecular types, such as proteins (e.g., PD-L1), carbohydrates (e.g., Sialyl Lewis X), lipids, or nucleic acids, depending on the particle type (EV or NVP). Detection of co-localization may be accomplished using proximity-dependent assays, such as the Proximity Ligation Assay (PLA), which generates a quantifiable signal (e.g., via qPCR or Next-Generation Sequencing (NGS)) only when the target biomarkers, bound by specific probes, are within close spatial proximity on the same particle. Alternative single-particle analysis techniques such as advanced flow cytometry or super-resolution microscopy may also be employed.

By analyzing these specific multi-marker co-localization signatures on individual particles, the methods described herein offer the potential for enhanced sensitivity and specificity compared to conventional diagnostic approaches for cancers, e.g., LUAD, such as imaging or single-biomarker tests. The methods of the present disclosure may be used for various clinical purposes, including screening of high-risk populations (e.g., smokers), differential diagnosis of cancer, early detection of cancer e.g., LUAD, and monitoring therapeutic response or disease recurrence.

The disclosure further encompasses kits containing the necessary reagents (e.g., capture agents, detection probes, enzymes) and systems (e.g., automated or microfluidic platforms) designed to perform the described particle capture and co-localization detection methods.

### BRIEF DESCRIPTION OF DRAWINGS

FIG. 1 is a schematic showing enrichment of extracellular vesicles and particles (EVPs) from a sample matrix using size-exclusion chromatography.

FIG. 2 is a schematic showing affinity reagents targeting cancer-associated biomarkers conjugated to magnetic beads as capture reagents and to oligonucleotides as detection probes.

FIG. 3 is a schematic showing capture of EVPs with antibody-functionalized magnetic beads and detection with complementary detection probes that hybridize and ligate when bound to the same EVP to generate a single-stranded DNA template detectable by qPCR.

FIG. 4 shows qPCR results from solution-phase ligation testing of detection probe DNA.

FIG. 5 shows assay signal from NCI-H2126 EVPs and buffer for biomarker combinations targeting CD63-positive and CD81-positive EVPs.

FIG. 6 shows reduction in assay signal following detergent exposure of captured NCI-H2126 EVPs.

FIG. 7 shows assay signal strength across a panel of cancer cell line EVPs for a four-biomarker assay design targeting lung adenocarcinoma EVPs.

FIG. 8 shows assay signal strength across detection probe configurations for NCI-H2126 EVPs and buffer only.

FIG. 9 shows linearity of assay signal for NCI-H2126 EVPs and NCI-H441 EVPs spiked into binding buffer.

FIG. 10 shows a representative super-resolution microscopy image of double-positive EVPs.

FIG. 11 shows a representative super-resolution microscopy image of triple-positive EVPs.

FIG. 12 shows a representative super-resolution microscopy image of quadruple-positive EVPs.

FIG. 13 shows abundances of double-positive, triple-positive, and quadruple-positive EVP subpopulations.

FIG. 14 shows absolute signal strength values for a three-marker combination and four-marker combination variants in a Lung Cancer Pool and a Healthy Donor Pool.

FIG. 15 shows ΔCt values comparing discrimination performance between a three-marker combination and four-marker combination variants.

FIG. 16 shows an overview of a biomarker combination screening process for development of a lung cancer-specific assay.

FIG. 17 shows assay signal distribution across lung adenocarcinoma cases and non-cancer controls.

FIG. 18 shows ROC curves summarizing discrimination performance for stage I, stage II, and stage III/IV lung adenocarcinoma cases versus non-cancer controls.

FIG. 19 shows assay signal as a function of tumor size and cancer stage among lung adenocarcinoma cases.

FIG. 20 shows correlation between model score and smoking history among non-cancer controls.

FIG. 21 shows a schematic representation of a captured extracellular vesicle (EV) with multiple distinct biomarkers. The EV is surrounded by three different probes, each used to detect one of the three distinct biomarkers on the surface of the EV.

FIG. 22 is a schematic diagram showing an exemplary proximity-dependent sequential ligation process involving the oligonucleotide portions of three distinct detection probes.

FIG. 23 shows a box plot and Receiver Operating Characteristic (ROC) curves comparing the sensitivity and specificity of distinct biomarker detection systems. Top panel depicts comparative qPCR Ct values for different biomarker combinations in cancer vs. control sample cohorts. Bottom Panel depicts Area Under the Curve (AUC) values of biomarker combinations.

### DETAILED DESCRIPTION

The present disclosure provides methods, compositions, kits, and systems for detecting conditions, such as Lung Adenocarcinoma (LUAD), in a subject by analyzing specific combinations of biomarkers associated with individual biological particles obtained from a biological sample. A biomarker refers to a distinct molecular indicator associated with a particular biological state or condition and may include proteins, lipids, carbohydrates, nucleic acids, or modifications thereof. There is a need for improved diagnostic tools for lung cancer, e.g., for early detection in high-risk populations (e.g., smokers) as current methods often lack sufficient sensitivity or specificity. The methods of the present disclosure address this need by providing a novel approach involving the analysis of multiple co-localized biomarkers on specific particle populations. This approach offers improved diagnostic accuracy compared to conventional methods. The method involves capturing target particles based on a first biomarker and subsequently detecting the co-localization of at least three additional biomarkers (e.g., three, four, five, six, or more biomarkers) on the same captured particle, resulting in analysis of at least four total biomarkers, using, for example, proximity-dependent assays such as the Proximity Ligation Assay (PLA). The methods described herein achieve high diagnostic accuracy suitable for early LUAD detection or monitoring.

Methods of the present disclosure may involve liquid biopsy techniques. Liquid biopsy is a minimally invasive technology that involves the analysis of biomarkers in bodily fluids (e.g., blood, urine, saliva) to detect, diagnose, and monitor diseases such as cancer. Liquid biopsy techniques avoid the need for invasive tissue biopsies by analyzing circulating analytes like ctDNA, CTCs, and various types of biological particles shed from tumors or affected tissues.

“Pan-EV capture” refers to capture strategies configured to isolate a broad or substantially non-selective population of extracellular vesicles and/or particles, without requiring that the capture biomarker be specific to a disease or pathological condition.

### Biological Particles

Biological fluids contain a complex mixture of particles derived from cells. The methods described herein are applicable to analyzing biomarkers on various types of such particles, including both Extracellular Vesicles (EVs) and Non-Vesicular Particles (NVPs).

Extracellular Vesicles (EVs) are vesicular particles enclosed by a lipid bilayer. EVs may be released from cells. EVs play roles in intercellular communication and waste removal, and their cargo (e.g., proteins, lipids, nucleic acids) may reflect the state of their cell of origin. Major types of EVs relevant to diagnostics include exosomes, microvesicles, apoptotic bodies, and oncosomes.

Exosomes are small EVs, about 30-150 nm, originating from the endosomal pathway. Microvesicles, or ectosomes, are larger EVs, about 100-1000 nm, budding directly from the plasma membrane. Apoptotic bodies are vesicles released during programmed cell death (i.e., apoptosis). Oncosomes are large EVs shed by cancer cells.

Non-Vesicular Particles (NVPs) are subcellular or supramolecular entities not enclosed by a continuous lipid bilayer. Examples include lipoproteins, protein aggregates, genomic aggregates, ribonucleoprotein complexes, exomeres, and supermeres. Lipoproteins, such as LDL, HDL, and VLDL, carry lipids and comprise lipids and proteins. Protein aggregates may include disease-associated aggregates or aggregated oncoproteins. Genomic aggregates are complexes involving DNA fragments. Ribonucleoprotein complexes are assemblies of RNA and proteins. Exomeres and supermeres are specific classes of nanoparticles lacking a lipid bilayer. Exomeres and supermeres may carry proteins, metabolites, and nucleic acids. Exomeres/Supermeres may be distinct populations of nanoparticles lacking a lipid bilayer found in bodily fluids, smaller than exosomes. Exomeres, about 35 nm, may contain metabolic enzymes and proteins related to hypoxia and glycolysis. Supermeres, larger than 50 nm, may overlap in size with small EVs and carry proteins involved in coagulation and immunity, along with lipids and nucleic acids.

Both EVs and NVPs can originate from various cells, including lung adenocarcinoma cells, via secretion, shedding, or release during cellular processes. Their composition can change in response to disease states like cancer, making them useful for the co-localization biomarker analysis described herein. A biomarker is considered “associated with” a particle if it is physically part of it, bound to its surface, contained within its structure, or located within its lumen or membrane (e.g., for EVs).

### Isolated Particles

Following isolation, analysis of the particle population, e.g., to determine whether EV or NVP, may be performed. Techniques such as Nanoparticle Tracking Analysis (NTA) or Dynamic Light Scattering (DLS) may provide information on size distribution. Electron microscopy (e.g., TEM, SEM) may provide morphological information (e.g., distinguishing vesicular from non-vesicular structures). Proteomic analysis (e.g., mass spectrometry) or Western blotting may confirm the presence of biomarkers (e.g., EV markers including tetraspanins CD9/CD63/CD81, or specific NVP components) and identify other associated molecules.

### Sample Collection and Preparation

The methods utilize a sample obtained from a subject. Suitable samples containing relevant particles include, but are not limited to, whole blood, plasma, serum, sputum, urine, saliva, cerebrospinal fluid (CSF), ascites fluid, pleural effusion, lymph, tears, and sweat. Samples are collected using standard clinical procedures (e.g., venipuncture for blood). Plasma or serum is used for analyzing circulating particles relevant to lung cancer and is isolated by centrifugation to remove blood cells. Sputum may be used for analyzing respiratory-derived particles relevant to lung cancer and is processed to remove cellular debris and reduce viscosity prior to particle isolation. Urine may be used for detecting bladder or kidney cancer markers, while saliva may be used for oral or head and neck cancer detection, and specific standardized collection protocols for those sample types are used. Additional centrifugation or filtration to remove large debris, may be performed. Samples may be analyzed fresh or stored, e.g., frozen (e.g., at −80 °C), ensuring particle and biomarker integrity until analysis. Other variables taken into consideration in sample collection and preparation include sample collection tube type, time between collection and processing, centrifugation protocols, and storage conditions (e.g., temperature, freeze-thaw cycles).

### Isolation of Target Particles

The methods of the present disclosure involve isolating a target population of particles (e.g., EVs and/or NVPs) from the biological sample based on a first biomarker associated with these particles (e.g., particles linked to LUAD). This biomarker serves as an anchor for capture.

In certain embodiments, the first biomarker is associated with the disease or condition being detected (disease-specific capture). In other embodiments, the first biomarker is not condition-specific and is used for pan-EV (agnostic) capture of a broad particle population, with diagnostic specificity conferred downstream by the multi-marker detection architecture.

Isolation uses a capture agent specific for this first biomarker. Capture agents include antibodies, antibody fragments, aptamers, lectins, lipid-binding reagents, or other specific ligands. The first biomarker may be a protein (e.g., EGFR, CEACAM5) or a carbohydrate moiety (e.g., SialylTn, Sialyl Lewis X).

Aptamers are short, single-stranded nucleic acid sequences (DNA or RNA) that can fold into complex three-dimensional structures capable of binding to proteins, small molecules, or cells. They are generated through an in vitro selection process (SELEX),

Immunoaffinity capture is a common technique, often using the capture agent immobilized on a solid support (e.g., magnetic beads, plates, microfluidic channels). For example, particles may be isolated from plasma using a first antibody immobilized on magnetic beads. The sample is contacted with the support, target particles bind, and unbound material is washed away, resulting in an isolated population of the target particles bound to the support. Other methods including size-exclusion chromatography, density gradient centrifugation, or precipitation may be employed as preliminary or alternative isolation steps.

Immunoaffinity Capture relies on the highly specific binding interaction between an antibody, or other ligand, and its target antigen (e.g., the biomarker). By immobilizing the antibody/ligand, it can be used to selectively “pull down” or capture molecules or particles bearing the target antigen from a sample.

### Disease-Specific Capture Embodiments

In certain embodiments, the capture agent binds a disease-associated biomarker, such as a LUAD-associated protein (e.g., EGFR or CEACAM5) or a disease-associated carbohydrate moiety (e.g., SialylTn or Sialyl Lewis X). In these embodiments, capture enriches a disease-associated particle subset, and detection of additional co-localized biomarkers yields high specificity.

### Pan-EV Capture

In other embodiments, the first biomarker is not specific to the condition, and the capture agent is configured to bind a biomarker broadly expressed across extracellular vesicles and/or particles, thereby enabling agnostic or pan-EV capture. In such embodiments, the capture step enriches a broad population of particles, and diagnostic specificity is derived from subsequent detection of co-localized biomarker combinations on individual particles.

Pan-EV capture strategies may include, but are not limited to:
(a) Immunoaffinity capture of pan-EV surface proteins, including capture via tetraspanins and related proteins broadly expressed on EV membranes, such as CD9, CD63, CD81, TSPAN1, TSPAN2, TSPAN3, and combinations thereof, and integrins of the ITGA and ITGB families.
(b) Carbohydrate-based capture, including capture using lectins, glycan-binding proteins, antibodies, aptamers, or other affinity reagents that bind carbohydrate or glycan structures broadly present on EV surfaces.
(c) Lipid-based capture, including capture via affinity to membrane lipids such as phosphatidylserine or glycolipids, using lipid-binding proteins, antibodies, or other lipid-binding moieties.
(d) Charge-based capture, including capture based on electrostatic interactions using ion-exchange beads, charged membranes, or surfaces configured to bind particles based on net surface charge.

Capture agents may be immobilized on magnetic beads, planar surfaces, microfluidic channels, membranes, or columns. In other embodiments, the capture agent may be provided in solution and subsequently immobilized or separated.

### Detection of Co-Localized Biomarkers

Following isolation, the captured particles are analyzed to detect individual particles that simultaneously display at least three additional biomarkers (e.g., a second, third, fourth, fifth, and sixth distinct biomarkers) in a co-localized manner. This multi-marker co-localization detection involving analysis of at least four total biomarkers (one capture and at least three, four, five, or six detected) is central to achieving high specificity for conditions such as LUAD.

In embodiments employing pan-EV or non-selective capture, the capture step serves to enrich a broad population of EVs/particles, while the determination of disease state is achieved by evaluating the simultaneous presence of multiple biomarkers on individual particles, such that diagnostic specificity resides in the downstream detection architecture (multi-marker co-localization) rather than in the capture reagent.

These additional biomarkers are selected for their combined diagnostic value for the target condition. They may be proteins (e.g., PD-L1, CEACAMs), carbohydrates (e.g., Sialyl Lewis X), lipids, or nucleic acids. These detected biomarkers may be of the same or different molecular types as each other and the capture biomarker. For instance, the capture biomarker and all the co-localized biomarkers detected may be proteins. In another embodiment, capture might be via a protein, while detection involves co-localization of another protein and a carbohydrate. In a certain embodiment, an additional biomarker is a carbohydrate/glycan structure, or a lipid/glycolipid such as phosphatidylserine or a ganglioside. The biomarker is detected via a method involving PLA. In another embodiment, an additional biomarker is a protein. The biomarker is detected via a method involving PLA. In another embodiment, the additional biomarker is an RNA molecule such as mRNA, miRNA, lncRNA, etc. The biomarker is detected via nucleic acid amplification methods (e.g., qRT-PCR).

In certain embodiments, one or more of the co-localized biomarkers detected on the captured particle is a different epitope of the first biomarker bound by the capture agent, such that the capture agent binds a first epitope of the first biomarker and one or more detection probes bind a second, distinct epitope of the same first biomarker on the same individual particle. For example, capture may be performed using an antibody that recognizes a first epitope of a tetraspanin (e.g., CD63 or CD81), and detection may be performed using one or more antibodies that recognize a second, distinct epitope of the same tetraspanin, optionally together with detection probes specific for at least two additional distinct biomarkers on the same individual particle. Such configurations yield a multi-marker co-localization signature in which at least one detected biomarker comprises a different epitope of the first biomarker.

Methods of the present disclosure for detecting co-localization may involve a Proximity Ligation Assay (PLA). This method involves the use of distinct probes specific for each of the multiple additional biomarkers being detected (i.e., at least three distinct probes corresponding to the three additional biomarkers). Each probe comprises a target binding moiety (e.g., an antibody or aptamer) linked to an oligonucleotide. The oligonucleotide may comprise a single-stranded portion. The oligonucleotide may comprise a sticky end. The method further involves contacting the isolated particles with the probes. If a particle displays the target biomarkers in close proximity, the corresponding probes will bind nearby. The proximity of the bound probes allows their single stranded portions of the attached oligonucleotides to hybridize. A nucleic acid ligase enzyme joins these hybridized single stranded portions, creating a ligated nucleic acid template. The method further involves detecting and quantifying the ligated template using methods such qPCR, dPCR, or DNA Sequencing (e.g., Next-Generation Sequencing, NGS). The resulting signal reflects the number of particles positive for the specific multi-marker co-localization signature.

In certain embodiments, pan-EV capture is performed using (i) immobilized anti-tetraspanin antibodies (e.g., anti-CD9, anti-CD63, anti-CD81) on magnetic beads, (ii) lectin-functionalized supports to bind broadly present EV glycans, (iii) lipid-binding capture agents targeting accessible phosphatidylserine, or (iv) ion-exchange supports configured to capture negatively charged particles. Following capture, the co-localization signature may be detected by PLA using three or more biomarker-specific probes, with optional scaling to four, five, or more probes using bridging oligonucleotide architectures described below.

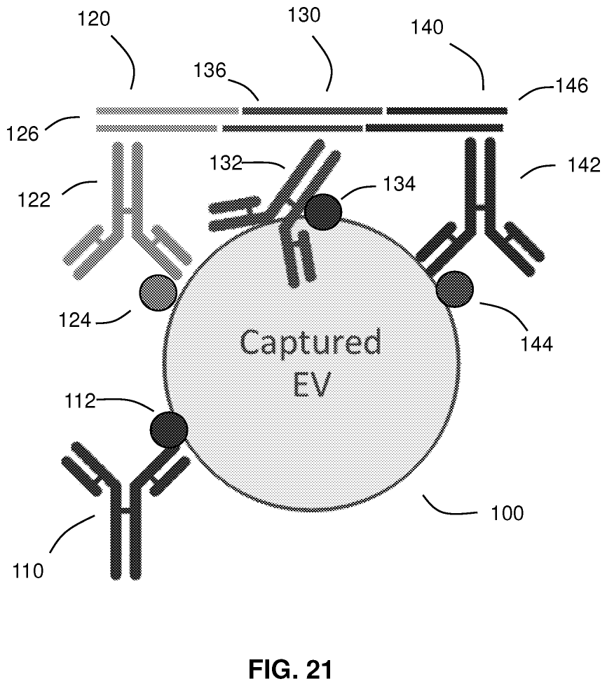

FIG. 21 shows an exemplary embodiment of a multi-biomarker detection strategy on a target nanoparticle 100 (e.g., an extracellular vesicle) is shown. The nanoparticle 100 displays a plurality of distinct surface biomarkers. A capture agent 110, which may be immobilized on a solid support (not shown) or be part of the detection complex, binds to a first biomarker 112 on the nanoparticle surface. Additionally, a first detection probe 120, a second detection probe 130, and a third detection probe 140 are shown. The first detection probe 120 comprises a first affinity reagent 122 specific for a second biomarker 124 on the nanoparticle 100, and is conjugated to a first nucleic acid oligonucleotide 126. The second detection probe 130 comprises a second affinity reagent 132 specific for a third biomarker 134 on the nanoparticle 100, and is conjugated to a second nucleic acid oligonucleotide 136. The third detection probe 140 comprises a third affinity reagent 142 specific for a fourth biomarker 144 on the nanoparticle 100, and is conjugated to a third nucleic acid oligonucleotide 146. The co-localization of biomarkers 112, 124, 134, and 144 on the same nanoparticle 100 brings the detection probes 120, 130, and 140 into close proximity, enabling the interaction of their respective nucleic acid oligonucleotides 126, 136, and 146. This arrangement, requiring four distinct co-localized biomarkers (e.g., one for capture, three for detection via ligation), forms the basis for a highly specific detection method.

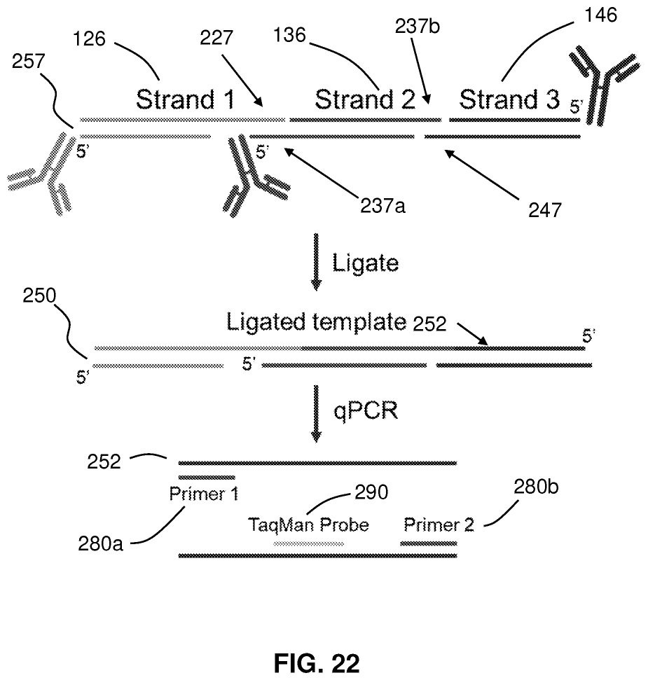

FIG. 22 depicts an exemplary mechanism for generating a nucleic acid template from co-localized detection probes. The first nucleic acid oligonucleotide 126 (from Probe 1) has a first single-stranded overhang 227. The second nucleic acid oligonucleotide 136 (from Probe 2, the central/bridging oligonucleotide) is designed with a first single-stranded overhang 237a at one end and a second single-stranded overhang 237b at its other end. Overhang 237a is complementary to overhang 227 of oligonucleotide 126. The third nucleic acid oligonucleotide 146 (from Probe 3) possesses a single-stranded overhang 247 that is complementary to overhang 237b of oligonucleotide 136. When the binders (122, 132, 142) bind their respective co-localized biomarkers, oligonucleotides 126, 136, and 146 are brought into proximity. This allows overhang 227 to hybridize with overhang 237a, and overhang 237b to hybridize with overhang 247, to form a double stranded complex 257. The top strand of the resulting double stranded complex 257 includes a first ligation junction (nick) between oligonucleotide 126 and oligonucleotide 136, and a second ligation junction (nick) between oligonucleotide 136 and oligonucleotide 146. Upon addition of a suitable ligase enzyme, these nicks on the top strand are sealed (i.e., ligated), resulting in the formation of a nucleic acid product 250. The top strand of the nucleic acid product 250 is ligated template 252. This ligated template 252 can then be amplified (e.g., by qPCR using primers 280a and 280b and detected using a detection moiety such as a TaqMan probe 290) and quantified, providing a measure of nanoparticles exhibiting the specific four-biomarker combination. The requirement for two distinct ligation events significantly reduces non-specific signal generation. This method can be extended to incorporate more detection probes and ligation events by using additional oligonucleotides with dual overhangs as further bridging elements.

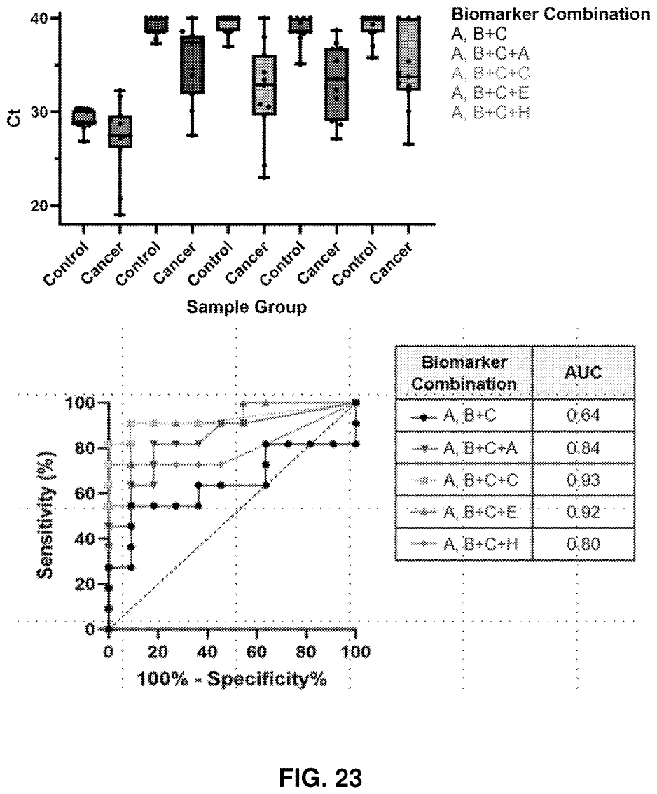

FIG. 23 illustrates the improved differentiation capacity achieved by requiring the co-localization of an increased number of biomarkers. Top panel shows a box plot of assay signals (e.g., qPCR Ct values, where lower values indicate stronger signals) from control groups (e.g., healthy individuals or no-target samples) and cancer patients. For a simpler biomarker combination (e.g., 3-marker system), there may be considerable overlap in signal distributions between the control and case cohorts. In contrast, for a higher-order biomarker combination (e.g., 4, 5, 6, or more biomarker combination), the signal distribution for the control group is shown to be tightly clustered at very low signal levels (or high Ct values, indicative of minimal background), while the cancer patients show a more distinct signal, resulting in improved separation between the cohorts. Bottom panel shows corresponding ROC curves. A ROC curve for the simpler combination may have a lower AUC value, indicating modest differentiation. ROC curves for the higher-order combinations are shown shifted towards the upper-left corner, possessing a significantly higher AUC value. This demonstrates an improvement in the ability to differentiate between cancer patients and control groups with high sensitivity and specificity when employing the higher-order co-localization methods, a result of the effective reduction in healthy background signal.

Alternative methods capable of detecting multiple markers on single particles may also be used. These include advanced multi-parameter flow cytometry, utilizing instruments and protocols adapted for small particle analysis (e.g., EV-flow cytometry), or high-resolution microscopy techniques (e.g., super-resolution fluorescence microscopy such as Stimulated Emission Depletion (STED) microscopy or platforms including the ONI Nanoimager), or electron microscopy with immunogold labeling. Other sensitive detection platforms may be used for detecting co-localized biomarkers on isolated particles. For instance, single-molecule detection techniques like Single Molecule Array (SIMOA) may be used to detect the co-capture of multiple detection probes on the same particle. Surface Plasmon Resonance (SPR) may be used to monitor sequential or simultaneous binding events on captured particles indicative of co-localization. Advanced mass spectrometry combined with chemical cross-linking may identify co-localized proteins. Spectroscopic methods such as Raman spectroscopy, enhanced with Surface-Enhanced Raman Scattering (SERS) tags conjugated to probes, may also provide molecular information related to co-localization.

Examples of carbohydrates and lipids that may be used in a biomarker signature include, but are not limited to Tn antigen, SialylTn (sTn) antigen, Thomsen-Friedenreich (T, TF) antigen, Lewis Y (also known as CD174) antigen, Lewis B antigen, Sialyl Lewis X (sLex) (also known as Sialyl SSEA-1 (SLX)) antigen, SSEA-1 (also known as Lewis X), beta1,6-branching, bisecting GlcNAc in a beta1,4-linkage, core fucosylation, Sialyl-T antigens (sT), Sialyl Lewis c antigen, Globo H, SSEA-3 (Gb5), SSEA-4 (sialy-Gb5), Gb3 (Globotriaose, CD77), Disialosyl-galactosylgloboside (DSGG), GalNAcDSLc4, Fucosyl GM1, GD1alpha, GD1a ganglioside, GD2 ganglioside, GD3 ganglioside, GM2 ganglioside, Lc3 ceramide, nLc4 ceramide, 9-O-Ac-GD2 ganglioside, 9-O-Ac-GD3 (CDw60) ganglioside, 9-O-Ac-GT3 ganglioside, Forssman antigen, Disialyl Lewis a antigen, Sialylparagloboside (SPG), Polysialic acid (PSA) linked to NCAM, Sialyl Lewis A antigen (also known as CA19-9), carbohydrate antigen 125 (CA-125), CanAg (glycoform of MUC1), Lewis Y/B antigen, Sialyltetraosyl carbohydrate, NeuGcGM3, GM3 (N-glycolylneuraminic acid (NeuGc, NGNA)-gangliosides GM3), phosphatidylserine, and combinations thereof.

Examples of proteins that may be used in a biomarker signature include, but are not limited to ALDH18A1, ALDHIA1, AP1M2, APOO, ARFGEF3, B3GNT3, BMPRIB, CDCP1, NT5E, CADM4, CANT1, CD24, CDH1, CDH17, CDH2, CDH3, CEACAM5, CEACAM6, CLDN3, CLDN4, CLGN, CLN5, CYP2S1, DSG2, ELAPOR1, ENPP5, EPCAM, EPHB2, FAM241B, FERMT1, FOLR1, FZD2, GALNT14, GALNT6, GJB1, GNG4, GNPNAT1, GOLM1, GPR160, GPRIN1, GRHL2, GSTP, HACD3, HE4, HS6ST2, IGSF3, ILDR1, KDELR3, KPNA2, KRTCAP3, LAMB3, LAMC2, LAPTM4B, LARGE2, LMNB1, LRRN1, LSR, MAL2, MARCKSL1, MARVELD2, MET, MUC1, MUC2, MUC4, MUC5AC, MUC13, NPTXR, NUP210, OPN, PARD6B, PMEPA1, PODXL2, PRAF2, PRSS8, RAB25, RAC3, RACGAP1, RAP2B, RCC2, RNF128, RNF43, RPN1, RPN2, SERINC2, SHISA2, SLC35A2, SLC39A6, SLC44A4, SLC4A4, SMIM22, SMPDL3B, SYAP1, SYT13, TMEM132A, TMEM238, TMEM9, TSPAN13, ULBP2, UNC13B, VTCN1, ABCA13, ADAM23, CYP4F11, HAS3, TMPRSS4, UGT1A6, PIGT, TOMM34, ACSL4, GPC3, ROBO1, SLC22A9, SLC38A3, TFR2, TM4SF4, TMPRSS6, ANXA13, CHST4, GAL3ST1, SNAP25, TMEM156, CLDN18, EPPK1, MUC13, OCLN, CFTR, GCNT3, ITGB6, ITGB6, LAD1, MSLN, TESC, TTR, LYPD6B, S100P, TMEM51, TNFRSF21, UPK1B, UPK2, ABCC4, FOLH1, RAB3B, STEAP2, TMPRSS2, TSPAN1, AP1S3, DSC2, DSG3, TMPRSS11D, KCNS1, LY6K, MUC4, SYNGR3, CELSR1, COX6C, ESR1, MUC1, ABCC11, ERBB2, SLC9A3R1, PROMI, PTK7, CDK4, DLK1, LMNB2, PCDH7, TMEM108, TYMS, SDC1, SLC34A2, BCAM, MUC16, ADAM17, ADAM28, ADAM8, ALCAM, AMHR2, AXL, BAG3, BSG, CCL2, CCL8, CCN1, CCN2, CCR5, CD274, CD38, CD44, CD47, CDH11, CETN1, CLDN1, CLEC2D, CLU, CSPG4, DKK1, DLL4, EGFR, ENPP3, EPHA10, ERBB3, FAP, FGF1, FGFR4, FLNA, FLNB, FLT4, FZD7, GFRA1, GM3, GPA33, GPC1, GPNMB, GUCY2C, HGF, ICAMI, IGFIR, ILIA, IL1RAP, IL6, ITGA6, ITGAV, KDR, KLK, KLK3, KLKB1, KRT8, LAG3, LGR5, LPR6, LY6E, MCAM, MDM2, MELTF, MERTK, MSTIR, MUC17, MUC5AC, MUCL1, NOTCH2, NOTCH3, NRP1, NT5E, PI4K2A, PLAC1, PLAUR, PLVAP, PPP1R3A, PRLR, PSCA, PVR, RET, SIPR1, SLC3A2, SLC7A11, SLC7A5, SPINK1, STAT3, STEAP1, TACSTD2, TF, TFRC, TGFBR2, TIGIT, TNC, TNFRSF10A, TNFRSF10B, TNFRSF12A, TNFRSF4, TNFSF11, TNFSF18, TPBG, VANGL2, VEGF, VEGFA, VEGFC, and combinations thereof.

Biomarkers may include lipids such as phospholipids and glycolipids associated with the particle, carbohydrates e.g., glycans on proteins or lipids, nucleic acids including various forms of intravesicular RNA (e.g., mRNA, microRNA, long noncoding RNA, circular RNA, etc.), or modifications thereof. The methods of the present disclosure involve capturing target particles based on a first biomarker and subsequently detecting the co-localization of multiple additional biomarkers on the same captured particle, using, for example, proximity-dependent assays like the Proximity Ligation Assay (PLA) for detecting protein biomarkers or nucleic acid amplification methods like qRT-PCR for detecting RNA markers.

The present disclosure includes embodiments of an assay platform and methods for the detection of target analytes. Such analytes may include, for example, disease-associated nanoparticles, such as extracellular vesicles (EVs), and involve analyzing combinations of multiple co-localized biomarkers. A primary challenge in such assays, for purposes such as, but not limited to, early cancer detection, is the management of “healthy background signals.” Such background signals can obscure positive signals and compromise assay performance. The embodiments described herein are able to achieve improved specificity and sensitivity by strategically minimizing this background. In certain embodiments, this is accomplished via detecting the simultaneous co-localization of an increased number of distinct biomarkers (e.g., three, four, five, six, or more) on a single nanoparticle, and employing probe designs to detect such co-localization events.

A key feature of the technologies provided herein is the discovery that increasing the number of distinct biomarkers required for signal generation on an individual nanoparticle exponentially decreases the statistical likelihood of such a complex set of co-localized biomarkers occurring by chance on nanoparticles from healthy individuals or other non-target sources. While assays requiring the co-localization of, for example, only two biomarkers may show considerable signal overlap between healthy and disease cohorts, increasing this requirement to three, and particularly to four or more co-localized biomarkers (e.g., a biomarker combination comprising one capture biomarker and three distinct detection biomarkers, or a biomarker combination comprising four distinct detection biomarkers), leads to a profound reduction in healthy background signal. It is understood that such a reduction can result in significantly lower background in control samples (e.g., “no EV” controls, negative cell line-derived EV samples) and improved accuracy (e.g., via increased separation between positive and negative cohorts, higher Area Under the Curve (AUC) values in Receiver Operating Characteristic (ROC) analysis) for systems requiring four or more markers compared to those requiring fewer markers. This substantial decrease in background is understood to play an important role in the high specificity (e.g., greater than 99.0%, or greater than 99.5%) and high sensitivity needed to detect low-abundance disease-associated nanoparticles.

In order to implement the detection of three, four, or more co-localized biomarkers, certain embodiments use certain designs for binder-nucleic acid conjugates (i.e., probes). In an exemplary embodiment for detecting a four-marker combination (e.g., using one capture agent and three distinct detection binders), probes are designed such that the nucleic acid oligonucleotides attached to the multiple detection binders must all be brought into close proximity on the surface of the target nanoparticle. Such proximity then enables one or more proximity-dependent reactions, for example, sequential ligation events (e.g., two ligation events for a three-detection marker system, which may involve bridging or splint oligonucleotides) to form a single, complete, and unique amplifiable nucleic acid product. The formation of this product is, in such embodiments, strictly dependent on the simultaneous presence and co-localization of all targeted biomarkers within the defined set of co-localized biomarkers. Such probe designs, in various embodiments, may feature binders, which may include, but are not limited to, antibodies, antigen-binding fragments thereof, aptamers, or other moieties capable of specific binding to target biomarkers (e.g., protein, glycan, or other molecular targets) on the nanoparticle surface, as are known in the art and described herein. Furthermore, these probe designs may feature nucleic acid components, which may comprise DNA, RNA, or analogs thereof, and may be designed with specific overhangs, sequences, or other features to ensure ordered and specific ligation (or other proximity-dependent reaction) only when all requisite probes are co-localized as intended. The methods described herein may, in some embodiments, detect five, six, or even more co-localized biomarkers by, for example, incorporating additional probes and engineering the nucleic acid components for more complex, sequential proximity-dependent reactions, demonstrating the scalability of the approach.

For instance, the nucleic acid oligonucleotide coupled to a first detection probe (Probe 1) may possess a single-stranded overhang at one end. The nucleic acid oligonucleotide coupled to a second, distinct detection probe (Probe 2) may be designed with two single-stranded overhangs, one at each end of its central double-stranded portion. The first overhang of Probe 2 is complementary to the overhang of Probe 1. The nucleic acid oligonucleotide coupled to a third, distinct detection probe (Probe 3) may possess a single-stranded overhang at one end, which is complementary to the second overhang of Probe 2.

This proximity allows the overhang of Probe 1 to hybridize with the first complementary overhang of Probe 2, and simultaneously allows the second overhang of Probe 2 to hybridize with the complementary overhang of Probe 3. This ordered assembly creates two adjacent nicks or gaps in one strand of the newly formed tripartite nucleic acid structure. The addition of a suitable ligase then results in two ligation events: one between Probe 1 and Probe 2, and another between Probe 2 and Probe 3. These sequential ligations form a single, continuous, and unique amplifiable nucleic acid product, the formation of which is strictly dependent on the simultaneous presence and co-localization of all three targeted detection biomarkers and by extension, the capture biomarker if used.

To detect combinations involving more than three detection biomarkers (e.g., four detection biomarkers for a five-marker total combination, or five detection biomarkers for a six-marker total combination), additional “internal” or “bridging” detection probes, each coupled to an oligonucleotide possessing two distinct single-stranded overhangs (i.e., one at each end), are used. For example, to detect four co-localized detection biomarkers, Probe 1 (single overhang) would ligate to a first bridging Probe (Probe 2, dual overhangs); Probe 2 would ligate to a second bridging Probe (Probe 3, dual overhangs); and Probe 3 would ligate to a final Probe (Probe 4, single overhang). This would result in three ligation events. In general, for detecting n co-localized detection biomarkers, n−1 ligation events would be required, often involving n−2 internal probes with dual overhangs, and two terminal probes each with a single overhang. This modular design allows for the systematic increase in the number of co-localized biomarkers required for signal generation.

It is contemplated that while increasing the number of required co-localized biomarkers generally enhances specificity, a potential trade-off may be an attenuation of the absolute signal generated from target nanoparticles. However, the significant improvement in the signal-to-noise ratio, resulting from the significant reduction in background signal, leads to overall superior assay performance, particularly for achieving high specificity. The optimal number of biomarkers for a given biomarker combination can be tailored for specific applications and desired performance characteristics.

In some embodiments, the capabilities of the platform are further enhanced by analyzing multiple distinct combinations of co-localized biomarkers from the same biological sample. Such multiplexed analysis can be achieved via several configurations. One such configuration involves assaying different biomarker panels in separate aliquots or reaction vessels (e.g., wells of a microplate). Another configuration involves using multiple distinct probe sets in a single reaction, wherein each set is designed to generate a uniquely identifiable reporter signal. Such unique identification may be achieved, for example, through DNA barcoding for subsequent Next-Generation Sequencing (NGS) readout, or by employing distinct fluorescent labels or other distinguishable tags; this approach can increase throughput and conserve sample volume. A further configuration involves combining multiple distinct probe sets wherein positive signals resulting from any targeted combination contribute to a common, aggregated readout signal, a strategy that may enhance overall detection sensitivity for the presence of any of the targeted biomarker combinations. These multiplexing approaches, when combined with the high specificity of each individual multi-marker combination, enable comprehensive and robust analysis from a biological sample.

While the methods and compositions described herein are advantageous for applications such as early cancer detection (including, e.g., multi-cancer screening, monitoring of treatment response, and detection of recurrence), reducing background signal and enhancing specificity through multi-marker co-localization are broadly applicable. Such applications may include, without limitation, the detection of nanoparticles or other relevant biological structures in the context of infectious diseases, neurodegenerative diseases, cardiovascular diseases, inflammatory conditions, organ transplant monitoring, and basic research. Kits comprising the necessary probes, reagents for proximity-dependent reactions (e.g., ligation enzymes and buffers), amplification reagents, and detection reagents, along with instructions for use, are also contemplated as embodiments of the present disclosure.

Correlation with Condition

Methods of the present disclosure may also involve correlating the detected co-localization signal with the presence, absence, or state of the condition (e.g., lung cancer) in the subject. The quantitative output from the detection step (e.g., qPCR Ct value, number of positive events from flow cytometry) is compared against reference values. This may involve comparison to a predetermined diagnostic threshold, established through clinical studies on patient cohorts with known disease status. Signals exceeding the threshold may indicate the presence of the condition. Alternatively, signals may be compared to those from control groups (e.g., healthy individuals, individuals with benign conditions) to assess differences.

High diagnostic specificity relies on the detection of multi-marker co-localization. Methods of the present disclosure include using appropriate controls. In certain embodiments, the methods include processing samples from confirmed healthy individuals and subjects with relevant benign conditions; using isotype controls or irrelevant binding agents in parallel with specific capture agents and probes; omitting key enzymes (e.g., ligase in PLA) to assess background signal; and including positive and negative control materials with known particle/biomarker compositions.

The quantitative results provided by the methods of the present disclosure allow for monitoring of disease progression or treatment response, for instance, by comparing results from longitudinal samples taken from a subject undergoing therapy. The methods may also be applied for screening asymptomatic subjects considered high-risk for a specific cancer type (e.g., based on genetic predisposition or family history).

In embodiments where multiple distinct co-localization signatures are measured simultaneously across different biomarker combinations (i.e., multiplexing), data analysis may involve normalization, statistical modeling, or machine learning algorithms (e.g., logistic regression, support vector machines, decision trees) trained on clinical datasets to integrate the multiplex data into a single diagnostic score or classification.

### Biomarker Selection and Validation Strategy

The selection of the set of biomarkers (e.g., one capture, plus at least three, four, five, or six co-localized detection markers) plays an important role in the methods of the present disclosure. Candidate biomarkers may be identified through discovery via comparative multi-omics (e.g., proteomics, glycomics, etc.) of particles from diseased vs. control subjects. Public databases and literature searches may also inform candidate biomarker selection. For example, in Lung Adenocarcinoma (LUAD), the methods involve capturing particles via an antibody to EGFR (Epidermal Growth Factor Receptor) and detecting co-localization of CEACAM5, PD-L1, and a specific glycoform of CK7 (Cytokeratin 7). In another embodiment, the method involves capturing via CEACAM5 and detecting co-localization of EGFR, Napsin-A associated protein/epitope, and PD-L1.

The specific combination of capture and the at least three detection markers (e.g., three, four, five, six, or more biomarkers) may be optimized for sensitivity and specificity for LUAD, and not for other lung cancer subtypes (e.g., squamous cell carcinoma) or benign lung conditions. Similar strategies may be applied to other cancers by selecting appropriate biomarkers. For example, CA-125, MUC1, CEACAMs, FOLR1, STn, or SLeX may be relevant for other adenocarcinomas like ovarian or colorectal cancer. Candidate biomarkers and their combinations may be validated using techniques such as Western blotting, ELISA, mass spectrometry, or preliminary co-localization assays on well-characterized clinical cohorts to confirm their association with the target condition, their presence on the target particles (EVs or NVPs), and their ability to provide diagnostic information when assessed in combination.

### Conditions for Detection

There is a need for better early detection and characterization tools for cancer. The methods are broadly applicable to cancers (e.g., lung, ovarian, pancreatic, breast, colorectal, etc.) and other diseases where specific multi-marker particle signatures exist.

Lung cancer is the leading cause of cancer death globally. Early detection significantly improves survival, but low-dose CT screening, while beneficial for high-risk individuals, has limitations regarding false positives and detection of indolent or aggressive disease. The methods of the present disclosure may involve non-invasive liquid biopsy tests that accurately detect early-stage LUAD and/or differentiate subtypes.

### Clinical Use

The methods described herein may have clinical uses that relate to lung cancer and other diseases. The methods may be used for screening in high-risk individuals (e.g., heavy smokers) alongside or as an adjunct to imaging e.g., low-dose CT, improving specificity. The methods may be used to identify LUAD at stages I/II when curative treatment is more likely. The methods may provide information on biomarker expression (e.g., PD-L1, EGFR status via protein co-localization) relevant to targeted therapy or immunotherapy selection. The methods may be used for tracking and monitoring treatment response or detecting minimal residual disease/recurrence earlier than imaging. The method may be optimized based on the specific intended use.

### Treatment Efficacy

In some embodiments, multiple samples are taken from a subject during and/or following therapy to determine a therapy response. The therapy may include hormone therapy, immunotherapy, chemotherapy, photodynamic therapy, radiation therapy, hyperthermia, stem cell transplant, surgery, and/or targeted therapy.

The sample may be taken over a time period such as 1 day, 2 days, 3 days, 4 days, 5 days, 6 days, 1 week, 2 weeks, 3 weeks, 4 weeks, 1 month, 2 months, 3 months, 4 months, 5 months, 6 months, 1 year, 2 years, 3 years, 4 years, or 5 years. In an embodiment, the samples are taken over a period of 100 days following the start of the therapeutic treatment. In certain embodiments, at least three samples are taken from the subject.

The samples are tested to measure a multi-marker co-localization signal. The biomarkers may be selected to track a co-localization signal associated with EV particles. The signal may be monitored over time to determine if a signal level is increasing, decreasing, or remaining approximately constant. A change in the signal level may be associated with a therapy response. If the signal level decreases, the therapy may be classified as effective. If the signal level does not decrease, the therapy may be classified as ineffective.

At least two samples showing a decrease in the signal level may indicate the therapy is effective. The therapy may be classified as effective if the signal level decrease is sustained for a period of time such as at least 1 week, 2 weeks, 3 weeks, 4 weeks, 1 month, 2 months, 3 months, 6 months, or 1 year.

In an embodiment, a subject with LUAD is monitored for a multi-marker co-localization signal from biomarker probes, as described below. The subject is scheduled to begin chemotherapy, and an initial sample is taken. The multi-marker co-localization signal is measured and recorded at a time T₀ prior to beginning the therapeutic treatment. Following the start of chemotherapy, at least two samples are taken from the subject at a T₁ and a T₂ respectively. In a preferred embodiment, at least three samples are taken from the subject at predetermined time intervals for measurements at time points T₁, T₂, and T₃ respectively.

The co-localization signal of each sample may be compared to identify a change in the co-localization signal level. If the signal level is decreasing for at least two samples taken following the start of the therapy, the therapy may be classified as effective in treating LUAD. If the signal is not decreasing, the therapy may be classified as ineffective in treating LUAD.

In some embodiments, the rate of decline in the co-localization signal may be calculated to generate an elimination rate, k. A higher rate of decline may indicate a greater efficacy in treating the disease. For example, an elimination rate of >0 may indicate the therapy is successful. An elimination rate of ≤0 may indicate the therapy is not successful.

In certain embodiments, an elimination rate ≥1 may indicate the therapy is effective. An elimination rate of 1>k>0 may indicate the therapy is marginally effective. An elimination rate of k≤0 may indicate the therapy is not effective.

The elimination rate may indicate a likelihood of recurrence, with an elimination rate of k≥1 indicating a low risk of recurrence. An elimination rate of 1>k>0 may indicate a high risk of recurrence. A testing interval for monitoring a patient may be determined, based in part, on the elimination rate, k, of the therapy. A lower elimination rate such as k<1 may indicate the subject requires frequent monitoring.

In another embodiment, a subject with ovarian cancer is monitored for a multi-marker co-localization signal. The biomarkers may include at least three, and preferably at least four, of carbohydrate antigen 125 (CA-125), human epididymis secretory protein 4 (HE4), osteopontin (OPN), vascular endothelial growth factor (VEGF), transthyretin (TTR), Aldehyde Dehydrogenase 1 (ALDH1), Folate Receptor Alpha (FOLR1), and Glutathione S-Transferase Polymorphisms (GSTP).

The co-localization signal may be monitored during therapy, such as chemotherapy. A decline in the signal level may be detected and measured over time using periodic sampling. The rate of decline may be calculated, such as a CA-125 elimination rate constant K (KELIM) score. A score of >0 may indicate the therapy is successful. A score of ≤0 may indicate the therapy is not successful.

### Disease Progression and Longitudinal Monitoring

In some embodiments, multiple samples may be taken during and subsequent to therapy for detecting and monitoring a multi-marker co-localization signal over time. The samples may be tested using a selected set of biomarkers associated with a disease such as cancer to determine and track a disease state. The biomarkers may be selected based on a medical history of the subject. For example, if the subject has previously been diagnosed with LUAD, the biomarkers selected may be selected for detecting a co-localization signal indicating the presence of LUAD.

The samples may be evaluated, and the co-localization signal level may be measured and recorded for longitudinal tracking. An increase in the signal level over time may be associated with disease progression such as a cancer progressing from Stage I to Stage II. A decrease in the signal level over time may be associated with successful treatment and/or disease remission.

The subject may continue to be monitored following therapy by taking samples at periodic intervals to assess the co-localization signal level. If the signal level remains below a predetermined threshold, the subject may be classified as in remission. If the signal level increases or exceeds the predetermined threshold, the subject may be classified as having disease recurrence. The subject may be tested using a set of biomarkers selected for a cancer sub-type as described below. The subject may be tested every 1 month, 2 months, 3 months, 6 months, 1 year, 2 years, 3 years, 4 years, 5 years, or 10 years.

The interval between samples may be determined based, at least in part, on an elimination rate calculated during therapy. If the elimination rate is ≥1, the interval between sampling may be longer than if the elimination rate is <1.

### Minimal Residual Disease

Following therapy, a small number of cancer cells may remain in the patient in a condition referred to as minimal residual disease (MRD). For example, following surgical removal of a tumor, a small number of cancer cells may remain around the surgical site. The cancer cells may remain while the patient is in remission and may not be detected with conventional screening or imaging tests. MRD detection is associated with cancer recurrence and may detect an increase in the number of cancer cells prior to the subject presenting symptoms. MRD may refer to detecting low levels of cancer cells in tissue or blood samples. MRD may detect as low as one cancer cell amongst a million healthy cells. A subject undergoing MRD monitoring may be tested at intervals of 1 month, 2 months, 3 months, 4 months, 5 months, 6 months, 7 months, 8 months, 9 months, 10 months, 11 months, or 12 months following treatment to monitor for recurrence.

A set of biomarkers may be selected based on a medical history of the subject for monitoring a co-localization signal to detect recurrence of the disease. The systems and methods of the disclosure allow detection of even small numbers of cancer cells, such as those associated with MRD, and distinguish signals from cells of interest, such as cancer cells, from healthy cells identified as healthy background noise. The biomarkers selected may be specific to a disease the subject has been treated for. The biomarkers may be selected to identify mutated cancer cells and detect the recurrence of the disease.

### Kits and Systems

Kits would contain reagents specific for certain applications, such as a LUAD detection. In a certain embodiment, the kit comprises a capture agent (e.g., anti-EGFR antibody on beads); at least three sets of detection probes (e.g., PLA probes for CEACAM5, PD-L1, CK7-glycoform); enzymes, buffer solutions, controls, and instructions. Systems may be used to automate the process (e.g., microfluidic systems/platforms) integrating the steps involving isolation of particles, performance of reactions, and detection of co-localized biomarkers.

Variations in specific biomarkers, the number of co-localized biomarkers detected (e.g., at least three are detected in addition to the capture marker), particle types (EVs or NVPs), reagents, detection methods, sample types, and target conditions are all contemplated within the scope of the invention described herein.

### Examples

Tumor-derived extracellular vesicles and particles (EVPs) represent an analyte class for early cancer detection. However, discrimination of tumor-derived EVPs from those shed by healthy tissues represents an analytical consideration. The present disclosure describes the design and characterization of a novel proximity ligation-based immunoassay which targets tumor-derived EVPs in plasma displaying four colocalized surface biomarkers. The present disclosure demonstrates the functionality of this approach in cancer cell line-derived EVPs and apply the method to lung adenocarcinoma (LUAD) detection. Importantly, the data indicate that requiring four colocalized cancer-associated biomarkers reduces interference from healthy EVPs versus a three-biomarker design, resulting in strong discrimination performance for several biomarker combinations. Using this approach, a prototype assay for LUAD detection was evaluated in a training set comprised of 382 plasma samples. The assay and trained classifier exhibited 45.6% stage I sensitivity, 83.3% stage II sensitivity, and 100% stage III/IV sensitivity at 90% specificity. Assay signal was significantly correlated with tumor size and uncorrelated with smoking history. These results demonstrate the utility of this platform for early-stage lung cancer detection.

Lung cancer is responsible for the most cancer-related deaths globally, with approximately 2.5 million new cases and 1.8 million deaths each year. Prognosis is generally good when detected at a localized stage (5-year survival rate of 64.7%); however, over 70% of cases are diagnosed at regional or distant stages when the 5-year survival rate is as low as 9.7%. As a result, early detection represents one of the most effective methods for reducing lung cancer-related deaths. To this end, annual screening by low-dose computed tomography (LDCT) has been shown to reduce lung cancer-associated mortality and is currently recommended for individuals considered at high risk based on their smoking history. Notably, the mortality benefit observed during the National Lung Screening Trial (NLST) was primarily attributed to the detection of lung adenocarcinoma (LUAD)—the most common histological subtype accounting for approximately 50% of newly arising lung cancer cases. Despite the effectiveness of annual LDCT screening, uptake remains low with an average compliance rate of approximately 17% among eligible individuals.

Blood-based lung cancer detection tests have been proposed to improve screening compliance and/or complement LDCT performance. Numerous prototype tests have been reported based on measurement of circulating tumor DNA (ctDNA), serum proteins, cell-free RNA, and other analytes. However, no test has yet demonstrated sufficient sensitivity for preclinical early-stage disease at high specificity in an asymptomatic patient population—an essential criterion for broad clinical adoption.

Extracellular vesicles and particles (EVPs) are an analyte class for early cancer detection. EVPs represent a family of nanoparticles shed by cells including exosomes, ectosomes, oncosomes, apoptosomes, and non-membranous particles. The abundance of tumor-derived EVPs (tEVPs) in circulation represents a significant advantage over ctDNA, with >10,000 tEVPs per mL of blood expected from a 1 cm tumor. Moreover, tEVPs contain a package of colocalized biomolecules which represent the composition of their cell of origin, including cancer-associated neoantigens, over-expressed proteins, and microRNAs. Despite the predicted abundance of tEVPs in circulation, discrimination of tEVPs from healthy tissue-derived EVPs poses a technical consideration. More than 99% of EVPs in blood originate from hematopoietic cells with the remaining <1% derived from healthy solid tissues, and tEVPs are expected to represent fewer than 0.01% of total EVPs in circulation during early stage disease. Moreover, tEVPs may be heterogeneous in their molecular cargo and contain biomarkers which overlap with EVPs shed by healthy cells. As a result, targeting tEVPs for early cancer detection requires an analytical technique which can interrogate millions of EVPs at the single particle level and minimize interference from EVPs derived from healthy tissues.

Previously reported work describes a novel immunoassay platform which detects EVPs displaying three colocalized surface biomarkers. This sandwich immunoassay involves an immunoaffinity capture step followed by a proximity ligation reaction to generate a DNA template detectable using qPCR. Based on this assay design, EVPs must contain all three target biomarkers to generate assay signal. The approach utilized this approach to build a “biomarker combination” (i.e., capture plus detection biomarkers) specific for ovarian cancer-derived EVPs and subsequently validated the assay in multiple clinical sample sets. This same strategy may be applied to other cancer types by swapping in one or more affinity reagents targeting new capture/detection biomarkers. Cancer-specific biomarker combinations may be rationally designed to detect tumor-derived EVPs while minimizing signal from healthy tissues and/or non-cancer conditions.

Herein, the present disclosure describes a modified immunoassay design that enables the detection of EVPs displaying four colocalized surface biomarkers. It is contemplated that that increasing the number of colocalized cancer-associated biomarkers required to generate an assay signal could reduce the healthy background signal and improve the signal-to-noise ratio of the resulting test, ultimately increasing specificity over a three-biomarker design. This modified assay design was thoroughly characterized using EVPs derived from lung cancer cell lines and clinical samples. The platform functionality and discrimination performance relative to the three-biomarker assay design are demonstrated. The results demonstrate reduced healthy background signal when four cancer-associated biomarkers are required for assay readout relative to three biomarkers, resulting in superior signal-to-noise for some biomarker combinations. Using this approach, the present disclosure describes a panel of biomarker combinations specific for lung adenocarcinoma and evaluates discrimination performance in a case-control study comprising 92 LUAD cases and 290 non-cancer controls. The results were used to train a classifier, and performance was estimated using cross-validation where it is observed discrimination for early-stage disease. The assay panel and associated classifier will be evaluated in future independent sample sets.

### Methods

### Cell Culture

NCI-H441, NCI-H2126, NCI-H520, BT-20, and Farage cells were purchased from ATCC (Manassas, VA). Cells are thawed, sub-cultured and maintained in complete growth media. NCI-H441, NCI-H520, and Farage were maintained in RPMI-1640 medium (30-2001; ATCC) and BT-20 were maintained in Eagle's Minimum Essential Medium (30-2003; ATCC). Both media were supplemented with 10% MaxSpec fetal bovine serum (A; Gibco). NCI-H2126 cells were maintained in DMEM:F (30-2006; ATCC) supplemented with 5% MaxSpec FBS, 1X ITS (13146-5ML; Sigma-Aldrich), 10 nM hydrocortisone (H0135-1 MG; Sigma-Aldrich), 10 nM beta-estradiol (E2257-1 MG; Sigma-Aldrich), and 1X GlutaMAX supplement (35050061; ThermoFisher). Sf9 insect cells (11496015; ThermoFisher) were maintained in Sf-900 II SFM media (10902088; ThermoFisher) in 125-mL shaker flasks in a 25 °C incubator protected from light, without CO2 and without humidification on an orbital shaker platform set at 125-150 RPM.

For production of EVPs, cells were passaged into T175 tissue culture flasks or 150 mm dishes in complete growth media until 60% confluence was reached. The flasks or dishes were then transitioned to complete growth media but supplemented with EVP-depleted fetal bovine serum (A2720801; ThermoFisher). Briefly, complete growth medium was aspirated, cells are washed twice with PBS, and media containing EVP-depleted FBS was added. After 72 hours, the conditioned medium containing EVPs from the cells was collected and cellular debris removed by centrifugation at 1500×g for 15 minutes at 21 °C, followed by filtration with a 0.2 mm SFCA bottle-top filter unit. The cleared conditioned media is transferred to 500-mL PETG bottles and stored at −80 °C until EVP purification.

### Concentration of Conditioned Media

For each cell line, 200 mL of conditioned media stored at −80 °C is thawed at 25 °C in a circulating water bath and then concentrated via sequential centrifugation using a Centricon Plus centrifugal filter with a molecular weight cut-off of 100 kDa. Briefly, 60 mL of media was added to a Centricon filter and concentrated by centrifuging at 4347×g in an Eppendorf 5910 Ri centrifuge for 20 min. The process was repeated until all conditioned media was concentrated. Concentrated media was recovered by inverting the filter into a collection cup and centrifuging at 1000×g for 2 min.

### Pooled Plasma Samples

Healthy donor and lung cancer plasma pools are prepared from samples sourced from ProteoGenex (Inglewood, CA). To prepare each pool, individual plasma aliquots are thawed at room temperature, combined and thoroughly mixed, split into 1 mL aliquots, and stored at −80 °C. The Healthy Donor pool consisted of 18 unique subjects each contributing 1-4 mL of plasma, while the Lung Cancer Pool consisted of 13 unique donors each contributing 4-11 mL of plasma.

### Individual Plasma Samples

EDTA plasma samples were shipped frozen on dry ice to Mercy BioAnalytics and stored at −80 °C prior to testing. Healthy donor plasma was collected in the United States from a Mercy BioAnalytics-sponsored prospective collection after approval by the WCG Institutional Review Board (number 20212722). All individuals participating in the healthy collection study provided written informed consent, and the study complied with the Health Insurance Portability and Accountability Act and the Declaration of Helsinki. Blood from each healthy donor was collected using two 10-mL collection volume K2EDTA tubes (catalog number 366643; Becton Dickinson). After collection, each tube was inverted six to eight times and centrifuged at the collection site (1500×g at room temperature for 15 minutes) within 60 minutes of collection to separate the plasma from the other blood components. The resulting plasma was split into 1-mL aliquots and frozen at −80 °C.

All other plasma samples are obtained from commercial sources. Chronic obstructive pulmonary disease (COPD), benign nodule, and LUAD plasma samples are sourced from ProteoGenex Inc. and collected in Moscow, Russia. Benign nodule and LUAD plasma samples are sourced from iSpecimen (Woburn, MA) and collected in the United States. The remaining LUAD plasma samples are sourced from Ontario Tumour Bank (Toronto, Ontario) and collected in Ontario, Canada.

### EVP Purification from Concentrated Conditioned Cell Culture Media or Human Plasma

EVPs were purified from 1 mL of concentrated conditioned media or K2/K3EDTA human plasma by size exclusion chromatography using the qEVoriginal/70nmGen 2 columns (ICO-70; Izon Science). Briefly, SEC columns were flushed according to the manufacturer's instructions. Samples are thawed at room temperature, vortexed, after which 1 mL was loaded onto the column and allowed to flow into the resin bed by gravity. The void volume was discarded after the addition of 1.4 mL PBS. EVPs were then eluted in 1.6 mL PBS and were used for downstream analysis.

Purified EVPs from conditioned media were aliquoted, quantified using either the Zetaview Classic S model nanoparticle tracking analysis (NTA) instrument as described previously or nanoflow cytometry (described below) and stored at −80 °C until use. EVPs from human plasma were analyzed immediately.

### Antibody Selection

All antibodies were commercially sourced. Multiple monoclonal antibodies are obtained for each biomarker target and screened for functionality in cell line-derived EVPs. The top antibody clones are selected for further optimization (bead conjugation density, detection probe working concentration) in cell line EVPs before downstream testing in clinical samples.

### Preparation of Conjugated Assay Reagents

Antibody-functionalized paramagnetic beads (capture reagents) and DNA-functionalized antibodies (detection probes) are prepared as described previously. The complementary ssDNA pairs 1A+1B, 2A+2B, and 3A+3B were annealed prior to conjugation according to a standardized protocol.

### Assay Protocol for the Measurement of Four Colocalized Biomarkers on Single EVPs

EVPs isolated from concentrated conditioned media or human plasma are diluted in a binding buffer (0.1% BSA, 0.1% Pluronic F-68, 100 μg/mL salmon sperm DNA (15632011; ThermoFisher) and 0.1% ProClin 950 (46878-U; SigmaAldrich)) and subjected to immunoaffinity capture with antibody-functionalized paramagnetic beads on a Thermomixer C (Eppendorf) at 21 °C and 1100 rpm for 2 hours. Beads containing captured EVPs are transferred to a probe solution containing optimal concentrations of DNA-conjugated detection antibodies in binding buffer (0.1% BSA, 0.1% Casein, 0.1% Pluronic F-68 and 100 μg/mL salmon sperm DNA) for 30 minutes at room temperature. Beads were subsequently washed three times in PBST (PBS+0.05% Tween-20) to remove unbound detection probes before being transferred into ligation reaction mix. The proximity ligation reaction is performed at room temperature for 20 minutes in a reaction mix containing 1X ligase buffer (66 mmol/L Tris-HCl, 10 mmol/L Magnesium Chloride Hexahydrate, 1 mmol/L Adenosine-5′-triphosphate disodium salt trihydrate, 1 mmol/L DL-Dithiothreitol, 7.5% PEG 6000), Tween-20 (0.5%), and T7 DNA ligase (4687.5 units/mL). The resulting beads containing bound EVPs with ligated DNA template are transferred to a qPCR plate with master mix (1X LightCycler® 480 Probes Master (04887301001; Roche), 500 nmol/L primers, 200 nmol/L TaqMan probe and 0.5 U/reaction Uracil-DNA Glycosylase). The DNA template was amplified and quantified with the LightCycler® PRO (Roche) qPCR system using the following amplification conditions: 50 °C for 2 minutes, 95 °C for 10 minutes, 40 cycles at 95 °C for 5 seconds, and 60 °C for 20 seconds.

### Solution-Phase Ligation and DNA Characterization

Appropriate strands of annealed detection probe DNA (0.5 nM) were mixed with T7 DNA ligase (4500 units) in 30 μL of 1X ligase buffer. Each ligation reaction is incubated at room temperature for 20 minutes. The resulting amplicon is diluted to 0.5 μM and amplified using either LightCycler 480 Probes Master mix for qPCR readout or Equinox Library Amplification Kit without primers (7K0021-384; Watchmaker Genomics) for amplicon characterization.

Amplified DNA from solution-phase testing and/or the entire assay workflow is diluted 1:10 in nuclease-free water, quantified with the Qubit 1X dsDNA HS Assay Kit (Q33230; Invitrogen), and characterized by capillary electrophoresis using the DNA Bioanalyzer Kit (Agilent; 5067-1504).

### Detergent Sensitivity of EVPs

SEC-purified EVPs are diluted in a EVP binding buffer and subjected to immunoaffinity capture for 2 hours under agitation at 21 °C. Beads containing captured EVPs were then transferred into a detergent solution containing Triton-X (0.075%) in 1×PBS and incubated for 30 minutes at 21 °C under agitation to induce membrane disruption. The beads were then transferred through the remaining assay protocol as previously described including detection probe binding, proximity ligation, and qPCR.

### Biomarker Profiling in Cells by Flow Cytometry

Flow Cytometry was used to evaluate single biomarker expression on live cells. Cultured NCI-H2126, NCI-H441, Farage, BT-20, and NCI-H520 cells are washed with DPBS and then trypsinized with TrypLE (12605036; Gibco). Trypsin was neutralized with complete media, cells were pelleted by centrifugation at 300×g for 5 mins and then resuspended in Recovery™ Cell Culture Freezing Medium (12648-010; Gibco). Live cells were stored in liquid nitrogen until use.

Frozen cells are thawed at room temperature, centrifuged at 300×g for 5 min and the freezing media was aspirated from cells. The cells are washed once with and resuspended in Antibody Dilution Buffer (1316S; Cell Signaling Technology). Cell viability is confirmed for all cell lines and live cells were seeded at 5e5 cells per well in a 96-well plate for antibody staining. PE-conjugated antibodies against CEACAM5, CEACAM6, MUC1 and sTn were added to live cells at a final concentration of 5 μg/mL in 100 μL volume of Antibody Dilution Buffer. PE goat anti-mouse IgG (405307; Biotium) is diluted 1:1000 in Antibody Dilution Buffer and used as an isotype control. Cells are incubated with antibody at 4 °C for 1 hour, pelleted by centrifugation and washed twice with 100 μL Antibody Dilution Buffer. Cells were then resuspended in 250 μL Antibody Dilution Buffer for flow cytometry analysis.

For data acquisition, gain settings of FSC 60, SSC 60, FITC 105, and PE 191 are applied to the CytoFLEX S (Beckman Coulter, Brea, CA) and the acquired data was analyzed using FlowJo software (FlowJo LLC, Becton Dickinson & Company, V.10.0). For each biomarker, live cells were first gated using FSC-H vs SSC-H plots after which a subgate of single cell population was created using the SSC-H vs SSC-A plot. The PE-conjugated isotype control was then used to establish antibody background and create a PE+gate. Median fluorescence intensity (MFI) was determined and background signal from unstained cells was subtracted to determine the relative abundance of each biomarker on single cells.

### Biomarker Profiling in EVPs by EV Flow Cytometry

Flow cytometry on EVPs is performed following the MIFlowCyt-EV guidelines. EVPs were first stained with the protein reactive dye, AzDye488-TFP ester (FP-1026; Vector Labs) at a final concentration of 50 mM in 100 mL volume on a HulaMixer (15920D; ThermoFisher) at room temperature for 2 hours. Unreacted dye was removed using Exo-Spin mini columns (EX; Cell Guidance Systems). 10 mL of protein-stained EVPs were each incubated in PE conjugated sTn (0.5 mg/mL), CEACAM5 (1 mg/mL), CEACAM6 (1 mg/mL) and MUC1 (1 mg/mL) for 2 hours at room temperature. All staining is performed in duplicate. Stained EVPs are diluted 1:1000 in PBS and 100 mL of diluted stained EVPs were loaded onto the CytoFLEX S flow cytometer.

The CytoFLEX S was configured for small particle analysis using Violet Side Scatter (VSSC) for detection. Rosetta calibration beads (Exometry BV) are diluted one drop in 560 μL H2O per the manufacturers' instructions, VSSC was used as the primary threshold and set to 100,000. The gain for all fluorescent channels was set to 1000.

For sample measurement, the scatter gain was set to 500 for VSSC, SSC, and FSC. The primary threshold and width parameter were set to FITC, with a manual threshold value of 1900. All other settings remained the same. All samples are measured for 120 sec at a flow rate of 60 mL/min.

Data for bead calibrators and all samples were analyzed using Rosetta Calibration v.6 and FlowJo v.10.0. Briefly, the Rosetta calibration bead file was opened in the Exometry software, and the calibration and calculations are performed using the core/shell Mie model with PBS as the medium and a particle core refractive index of 1.38, a particle shell refractive index of 1.48, and a particle shell thickness of 6 nm. Size distributions were then exported to the appropriate FCS folder from the CytoFLEX to add a size parameter to the files, which were then loaded into the FlowJo software.

For analysis, a time gate was first established by gating out the first 20 seconds of measurement to exclude swarming effects. VSSC-H was then plotted (y-axis) versus the FITC-A (x-axis) to establish the protein-positive EVP gate for particles of interest. To identify the subpopulation of PE-positive EVPs, a gate was established using SF EVPs (negative control) on a plot of diameter (y-axis) versus PE-H (x-axis). The time gate, protein+EVP, and antibody-PE+EVP gates were then applied to all samples to determine biomarker positivity for each cell line EVPs.

To determine EVP stock concentration, the number of protein-positive EVPs was divided by 100 mL, which was the total volume measured on the flow cytometer. The resulting EVP/mL was multiplied by 20,000 to account for the 1:1000 dilution prior to flow cytometry measurement, the 5-fold dilution of EVP during antibody staining, and the 4-fold loss of EVP during total particle staining and cleanup with size exclusion columns.

### Super-Resolution Microscopy

Biomarker colocalization on single EVPs is evaluated with direct stochastic optical reconstruction microscopy using an ONI Nanoimager S and an EV Profiler Kit [Oxford Nanoimaging Inc. (ONI), Oxford, UK]. The top-performing capture and detection antibodies used in the EVP assay were conjugated to fluorophores for imaging using Lightning-Link kits from Abcam as follows: MUC1-Atto 488 (ab; Cambridge, UK), CEACAM6-Alexa Fluor 568 (ab; Cambridge, UK), and CEACAM5-Alexa Fluor 647 (ab; Cambridge, UK). Anti-sTn was biotinylated for EVP capture using Biotin Conjugation Kit (Fast, Type B)-Lightning-Link from Abcam (ab; Cambridge, UK). 10 μL each of SEC enriched NCI-H2126 EVPs are captured on imaging slides with the biotinylated sTn capture using the manufacturer-provided protocol. Briefly, the microfluidic imaging chip was functionalized with biotinylated anti-sTn (2.5 μg/lane) followed by a 75-minute incubation with EVPs using 0.02 μM filtered 1×PBS as a control. Post capture, EVPs were stained in ONI staining solution containing antibody cocktail consisting of 0.5 μg/mL each of anti-CEACAM5-AF, anti-CEACAM6-AF and anti-MUC1-Atto-488. The stained EVPs are washed; the chip was then prepared according to the manufacturers' instructions and imaged. A 30-millisecond exposure time was used to collect 3000 frames each at 640, 561, and 488 nm wavelength with laser power intensities of 38%, 30%, and 74% respectively on the CODI AutoEV imaging platform (https://alto.codi.bio, release date June 2023).

The Nanometrix software application (v.7.7.55, Nanometrix Ltd, Oxford, UK, www.nanometrix.bio) was used for batch image analysis. sTn-captured EVPs were defined as clusters identified by a density-based clustering (DBSCAN) algorithm with at least 20 localization events from one or more biomarker channels. At least 5 localizations of an individual biomarker channel were required to be classified as biomarker-positive. Clusters with an effective diameter <30 nm, fewer than 20 localizations or circularity <0.3 were discarded.

### Data Processing

Each clinical sample was processed via SEC and split equally across all biomarker combinations of interest. Data are presented as qPCR Ct. Wells with no signal were set to 40 Ct, representing no signal while allowing these data to be used. Experiments involving clinical samples were run in duplicate where the two technical replicates were collapsed to the maximum value for a conservative estimate of signal for downstream analysis. All other experiments were run with a variable number of replicates as specified in the text.

### Classifier Development

A LUAD-specific assay panel is developed comprising six unique biomarker combinations. This resulted in six features (i.e., Ct values) per sample which were combined into a single risk score using a trained classifier. Model development, evaluation, and presentation of results were based on the same set of samples in a leave one out cross validation with per-fold training. Three modeling techniques were built in R version 4.4.0 and evaluated: Random Forest, Elastic Net, and XGBoost without extensive hyperparameter tuning to limit overfitting. Each model is evaluated using a leave-one-out approach and Elastic Net is selected as the final model based on maximizing area under the curve and discrimination of early-stage lung adenocarcinoma from healthy individuals and individuals with benign tumors.

Final model scores for all samples were generated with Elastic Net (glmnet package version 4.1-8) with leave one out approach using the caret package (version 7.0-1) and the stats package (version 4.4.0) with “lamda.min” penalty parameter. For the purpose of model development and cross-validation, cases included all LUAD samples (n=92) and controls consisted of healthy individuals with a smoking history (current and former), healthy individuals without a smoking history, chronic obstructive pulmonary disease (COPD) patients, and individuals with benign tumors (n=290).

The threshold for binary classification of cancer samples was calculated at 90% specificity using the full set of controls (n=290) with their cross-validated model scores from Elastic Net. A cutoff of 90% specificity is selected to approximate the baseline specificity of low-dose CT imaging as interpreted by Lung-RADS.

### Statistical Analysis

Confidence intervals for AUC and sensitivities at 90% specificity were calculated with the ci.auc and ci.se functions respectively from the pROC package (version 1.18.5). Stage 3 lung cancer group had only 6 samples, far from enough for central limit theorem assumptions, so Wilson confidence intervals for sensitivity were calculated using wilson.ci from the fastR package (version 1.2.5).

Correlations between model scores and clinical variables including tumor size, smoking pack-years, sex, and age were calculated using Spearman correlation for continuous variables and non-parametric tests including Wilcoxon rank-sum test for discrete variables. Cell line gene expression data are obtained from the Cancer Cell Line Encyclopedia via the DepMap portal.

### Results

### Detection of EVPs Containing Four Colocalized Biomarkers

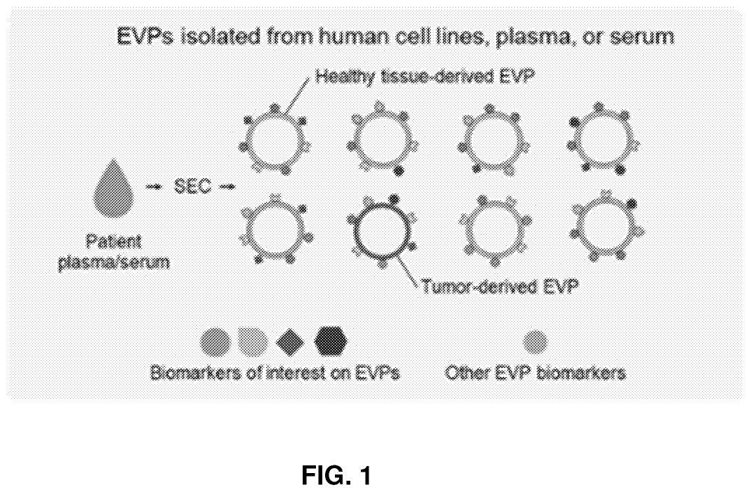

FIGS. 1-3 provide a schematic of the assay workflow enabling detection of EVPs containing four colocalized surface biomarkers. Briefly, EVPs are enriched from human plasma or serum using size-exclusion chromatography followed by an immunoaffinity capture step targeting a single membrane-associated surface biomarker. Captured EVPs are subsequently interrogated using three complementary DNA-antibody conjugates (i.e., detection probes) which target three additional surface biomarkers. These three detection probes can hybridize when bound to the same EVP and form a single DNA template through a proximity ligation reaction. The resulting full-length DNA template is detected via qPCR.

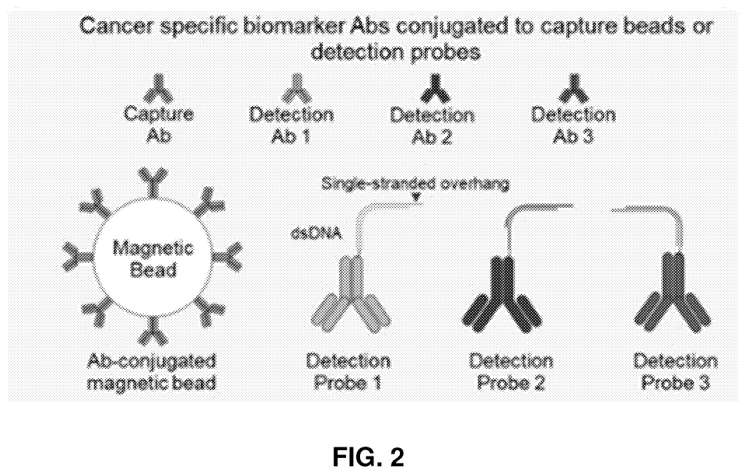

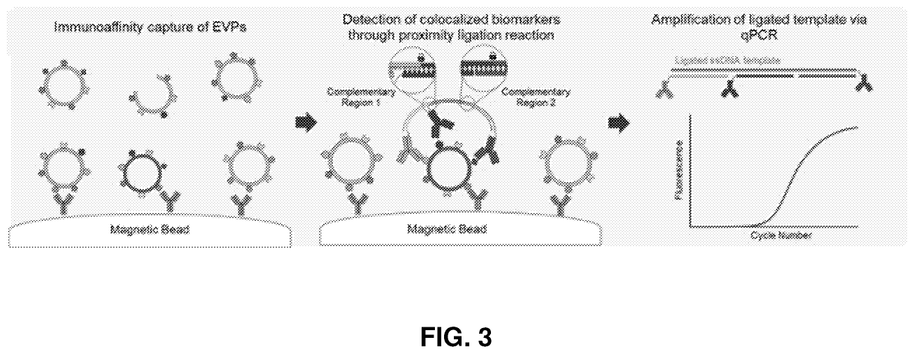

The DNA design for the detection probes is outlined in FIGS. 2-3. Each detection probe contains double-stranded DNA (dsDNA) with complementary single stranded overhangs on one (Probes 1 and 3) or both (Probe 2) ends. The resulting DNA complex includes two complementary regions pertaining to the Probe 1-Probe 2 junction and Probe 2-Probe 3 junction (FIG. 3). The sequences within these junctions are unique to lock the orientation of the complex and prevent competition between detection probes. Upon hybridization and subsequent ligation of the DNA complex, a single stranded DNA (ssDNA) template is generated for qPCR detection. The qPCR primers and fluorogenic hydrolysis probe require all three detection probes to be ligated together to generate detectable signal.

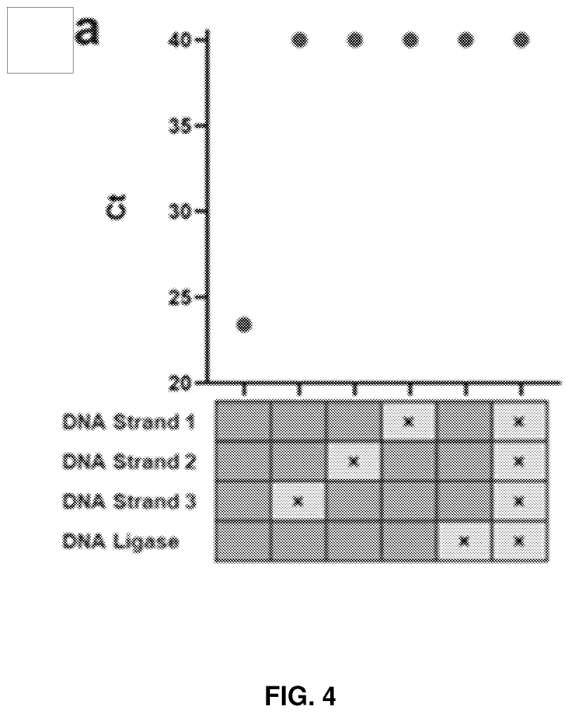

To demonstrate functionality of the DNA design, a solution-phase experiment was performed in which unconjugated detection probe DNA was mixed at various ratios and ligated. The resulting mixtures were subsequently assessed using qPCR. The results in FIG. 4 confirm robust qPCR signal (23.5 Ct) when all three DNA strands were mixed in the presence of DNA ligase. Conversely, no detectable signal was observed when at least one DNA strand was missing or when ligase was not added. The amplification product is characterized using capillary electrophoresis where it is observed a single peak at the expected DNA length of 128 base pairs. Moreover, the qPCR efficiency of the reaction is measured to be 101.2%.

An analogous experiment was performed to confirm functionality of the DNA-antibody conjugates within the entire assay workflow. All three strands of detection probe DNA were conjugated to monoclonal antibodies targeting the tetraspanin surface markers CD63 and CD81. EVPs from the LUAD cell line NCI-H2126 were isolated and characterized in the assay using configurations targeting a single biomarker (e.g., CD63-targeted capture, CD63-targeted detection). Based on this assay design, EVPs must display four distinct copies of CD63 or CD81 to generate signal. NCI-H2126 cells and EVPs are confirmed to be positive expressers of CD63 and CD81 using flow cytometry.

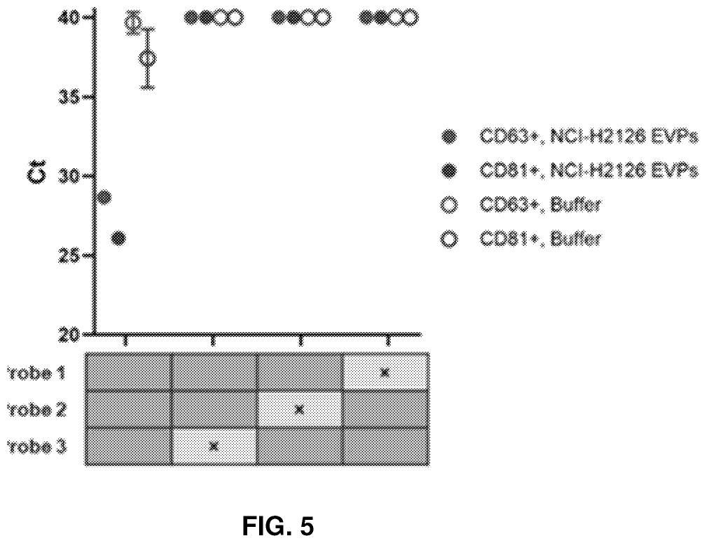

The results in FIG. 5 demonstrate that the assay is functional when all three detection probes are added. Specifically, strong assay signal was observed (Ct values≤30) for NCI-H2126 EVPs across both CD63- and CD81-targeted biomarker combinations, while reduced or no signal was generated when no EVPs were added (Buffer). In addition, assay signal was eliminated when at least one detection probe was omitted from the workflow, consistent with the results from solution-phase testing.

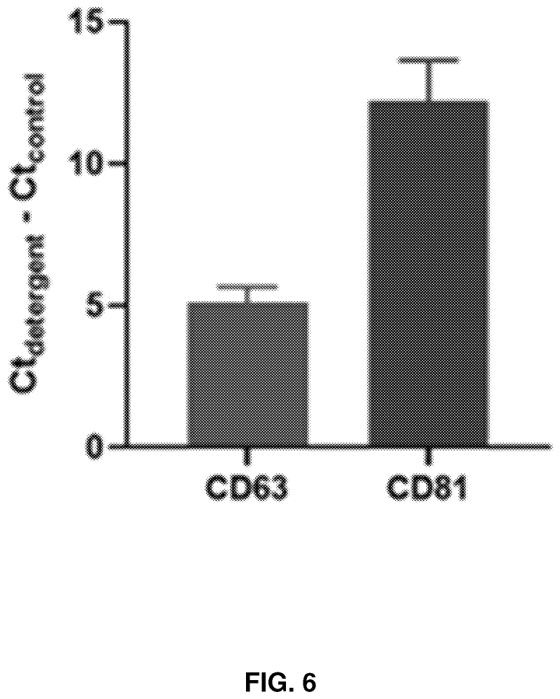

To confirm that biomarker colocalization is required for signal generation, the change in signal strength was measured upon exposure to a lysing detergent. To do this, the experiment is repeated the experiment above in which NCI-H2126 EVPs are captured using anti-CD63 and anti-CD81 beads. Captured EVPs were subsequently exposed to 0.075% Triton X-100 to disrupt the lipid bilayer and physically separate colocalized biomarkers. Following detergent exposure, the magnetic beads are transferred through the remaining assay workflow. The results in FIG. 6 demonstrate significant signal reduction (ΔCt>5) upon detergent exposure, consistent with a disruption of biomarker colocalization upon EVP lysis.

### Selection and Characterization of Lung Cancer-Specific Biomarkers

Lung adenocarcinoma-specific biomarkers were identified based on a custom biomarker discovery pipeline. Briefly, candidate genes are selected based on differential expression between LUAD tumors and healthy tissues and filtered for membrane localization based on Uniprot annotations, the abundance was then simulated of biomarker combinations in circulation for individuals with and without LUAD. Abundance estimates were generated based on biomarker expression levels within each tissue and estimated organ contributions to the EV levels in blood. Biomarker combinations were ranked based on simulated discrimination performance, and top performing combinations/biomarkers were identified for further evaluation. This process was supplemented by a literature review and analysis of additional proteomic/glycomic data sets to nominate biomarker candidates.

Based on the process outlined above, the following are selected CEACAM5, CEACAM6, MUC1, and sialyl-Tn (sTn) antigen for proof-of-concept testing. CEACAM5 and CEACAM6 are both members of the carcinoembryonic antigen cell adhesion molecule (CEACAM) family of proteins. They are GPI-anchored glycoproteins overexpressed in a variety of cancer types including LUAD and play a role in cell adhesion, tumor progression, and metastasis. MUC1 is a transmembrane mucin expressed on the apical surface of epithelial cells where it contributes to the formation of a protective mucosal layer. MUC1 is overexpressed and hypoglycosylated in LUAD and other cancer types, resulting in cancer-associated glycoforms which have been targeted for diagnostic and therapeutic applications. Overexpressed MUC1 has been shown to trigger signaling pathways which promote tumor growth and proliferation. Sialyl-Tn antigen is a truncated O-linked carbohydrate formed by sialylation of the Thomsen-nouveau (Tn) antigen by the enzyme ST6GALNAC1. sTn is overexpressed by several epithelial cancers including lung, and is found at higher levels in LUAD relative to other lung cancer subtypes. Overexpressed sTn promotes immune evasion and metastasis, and is associated with poor prognosis across several cancer types.

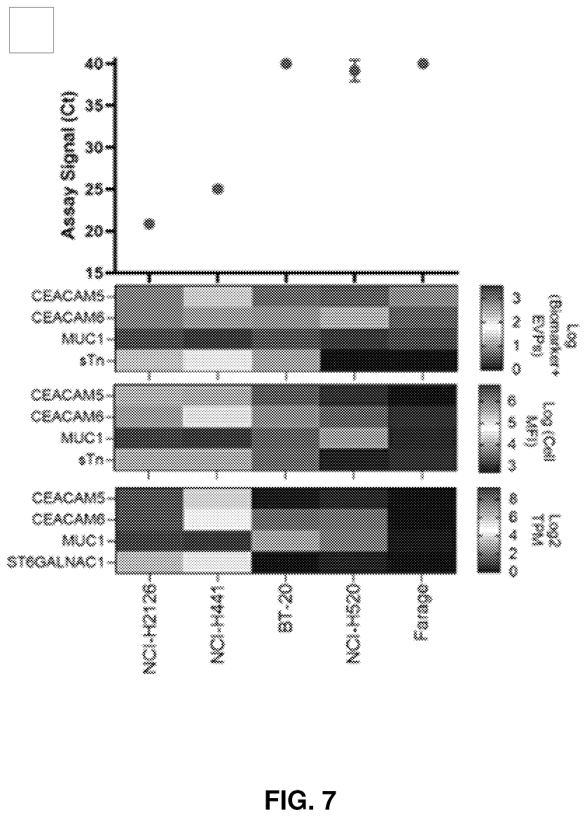

All four LUAD biomarkers are evaluated in a panel of human cancer cell lines which exhibit a range of expression levels. The cell lines NCI-H2126 (lung adenocarcinoma), NCI-H441 (lung adenocarcinoma), NCI-H520 (lung squamous cell carcinoma), BT-20 (breast cancer), and Farage (non-Hodgkin's B cell lymphoma) are characterized using flow cytometry to assess the expression of each biomarker at the cellular and EVP levels. The results are provided in FIG. 7 alongside previously published gene expression values. It is observed strong correlations between published gene expression and experimentally determined protein/carbohydrate expression, as demonstrated by Spearman correlation coefficients of 0.7-1.0. Moreover, the LUAD cell lines NCI-H2126 and NCI-H441 exhibited positive expression across all four biomarkers, while the other cell lines exhibited low/no expression for at least one marker. Particle size distributions of isolated EVPs were similar across all five human cancer cell lines as determined by EV flow cytometry.

### Application of Four-Biomarker Assay Design to Lung Cancer-Derived EVPs

To demonstrate detection of lung cancer-derived EVPs, the present disclosure describes an assay configuration targeting CEACAM5/CEACAM6/MUC1/sTn-positive EVPs and applied it to the cancer cell line panel described above. EVPs from each cell line were tested at an equivalent concentration of 8e7 particles/mL using sTn antigen as the capture biomarker and CEACAM5/CEACAM6/MUC1 as the detection biomarkers. The results in FIG. 7 demonstrate strong assay signal exclusively for cell lines which exhibit positive expression for all four biomarkers. Specifically, robust signal was observed for NCI-H2126 (CEACAM5+/CEACAM6+/MUC1+/sTn+) and NCI-H441 (CEACAM5+/CEACAM6+/MUC1+/sTn+) EVPs, while no signal was observed for NCI-H520 (CEACAM5−/CEACAM6+/MUC1+/sTn−), BT-20 (CEACAM5−/CEACAM6+/MUC1+/sTn−), and Farage (CEACAM5−/CEACAM6−/MUC1−/sTn−) EVPs. Signal from NCI-H2126 EVPs was stronger than NCI-H441 EVPs, likely driven by increased expression of CEACAM5 and CEACAM6 as measured by EV flow cytometry (FIG. 7). Moreover, assay signal was eliminated when at least one detection probe was omitted from the assay, consistent with FIG. 5.

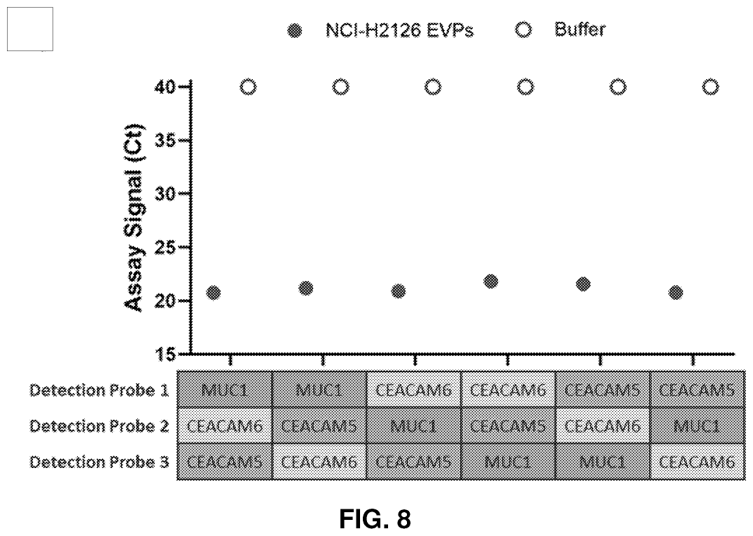

Among the cell lines positive for all four biomarkers, significant signal attenuation was observed (ΔCt>5) upon exposure to lysing detergent, suggesting that assay signal is derived from colocalized biomarkers on membranous particles. Interestingly, assay signal was stable upon varying the order of detection probes (e.g., CEACAM5/CEACAM6/MUC1 vs. CEACAM5/MUC1/CEACAM6; six total configurations), with a maximum Ct shift of 1.08 for NCI-H2126 EVPs (FIG. 8). This result suggests that steric factors exhibit a negligible impact on assay signal for this biomarker combination.

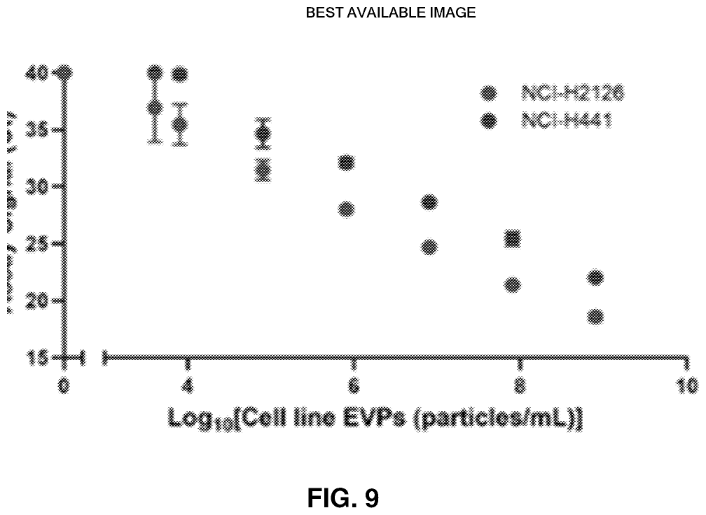

Assay linearity was assessed by performing a dilution series of NCI-H2126 and NCI-H441 EVPs spiked into binding buffer and characterizing the samples using the LUAD-specific biomarker combination described above. The results shown in FIG. 9 demonstrate linear assay signal within the concentration range of 8e3 and 8e8 EVPs/mL. No signal was detected in unspiked healthy plasma. Moreover, do not observe signal saturation at elevated EVP concentrations. The slopes of the linearity curves were-3.239 for NCI-H2126 EVPs and −3.196 for NCI-H441 EVPs, implying overall assay efficiencies of 103.5% and 105.5%, respectively.

Assay reproducibility is evaluated by measuring repeat sample aliquots across 12 distinct assay runs. Samples included NCI-H2126 EVPs spiked into healthy plasma at three concentrations pertaining to high, medium, and low signal, respectively. The spiked samples are prepared to obtain NCI-H2126 EVP concentrations of 8e7, 8e4, and 0 EVPs/mL following SEC purification. The observed inter-assay variability was low as demonstrated by coefficients of variation (CVs) less than 2% across all three spike-in concentrations.

### Orthogonal Validation of Biomarker Colocalization Using Super-Resolution Microscopy

To further characterize the functionality of the assay design described herein, visualization is performed on four-biomarker-positive EVPs using super-resolution microscopy. NCI-H2126 EVPs are captured onto microfluidic chips using an anti-sTn antibody. Captured EVPs were subsequently interrogated using fluorescently labeled detection antibodies targeting CEACAM5, CEACAM6, and MUC1. Each detection antibody was conjugated to a distinct fluorophore to enable independent measurement of expression level. The resulting images were interpreted using custom analysis software to identify EVP clusters and assess the biomarker positivity of each.

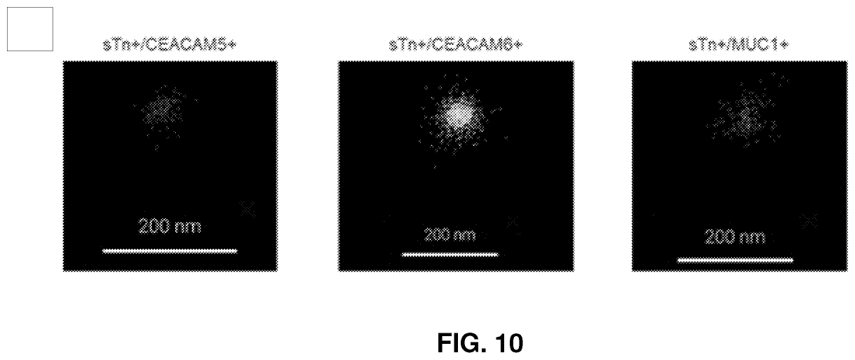

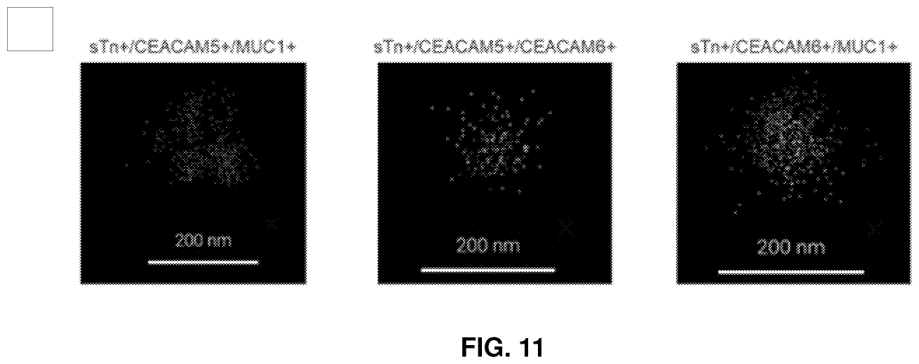

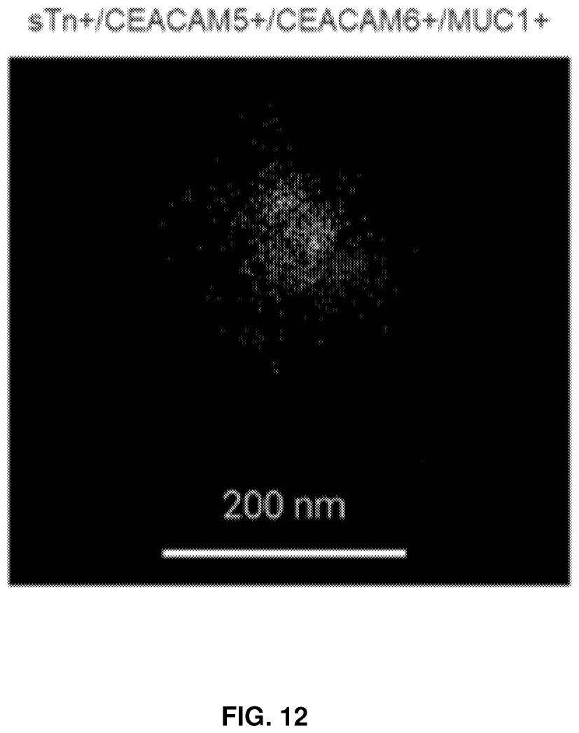

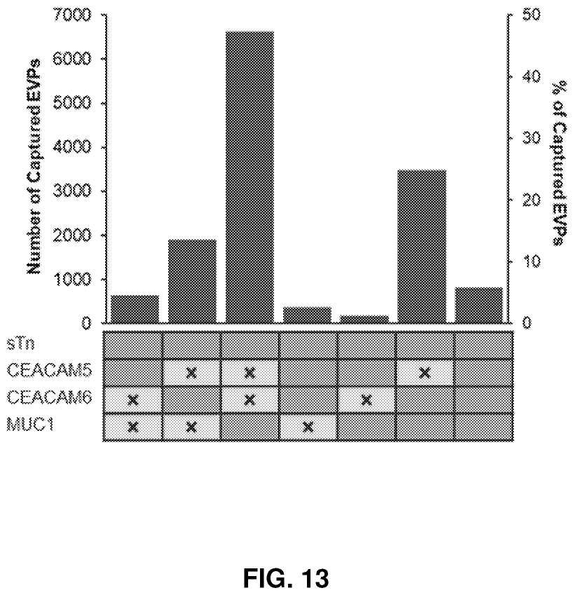

FIGS. 10-12 provide representative images of NCI-H2126 EVPs displaying two (FIG. 10), three (FIG. 11), and four (FIG. 12) surface biomarkers. Analysis was performed on more than 13,000 EVP clusters over 20 different fields of view to assess the overall abundance of double/triple/quadruple-positive EVPs. The abundance of EVP subpopulations varied by a factor of 40, as shown in FIG. 13. The most abundant species were sTn+/MUC1+/CEACAM5−/CEACAM6− and sTn+/CEACAM6+/MUC1+/CEACAM5− which accounted for 47.3% and 24.9% of total visualized clusters, respectively. Quadruple-positive EVPs (sTn+/CEACAM6+/MUC1+/CEACAM5+) represented 5.9% of total clusters and were the fourth most abundant subpopulation.

### Evaluation of Discrimination Performance for 3-Biomarker and 4-Biomarker Combinations

The discrimination performance of the 3-biomarker and 4-biomarker assay designs is compared in clinical samples. To do so, screening is performed on one lung cancer plasma pool and one healthy donor plasma pool against a set of biomarker combinations adopting the 3-marker and 4-marker configurations. This set included the 3-biomarker combo CEACAM5/CEACAM6/sTn as well as 4-biomarker variants built off the same motif (e.g., CEACAM5/CEACAM6/sTn/X). Biomarkers evaluated in the X position were identified using the process described above and included CDCP1, CEACAM5, CEACAM6, EPCAM, FOLR1, MSLN, MUC1, MUC16, NT5E, and TACSTD2.

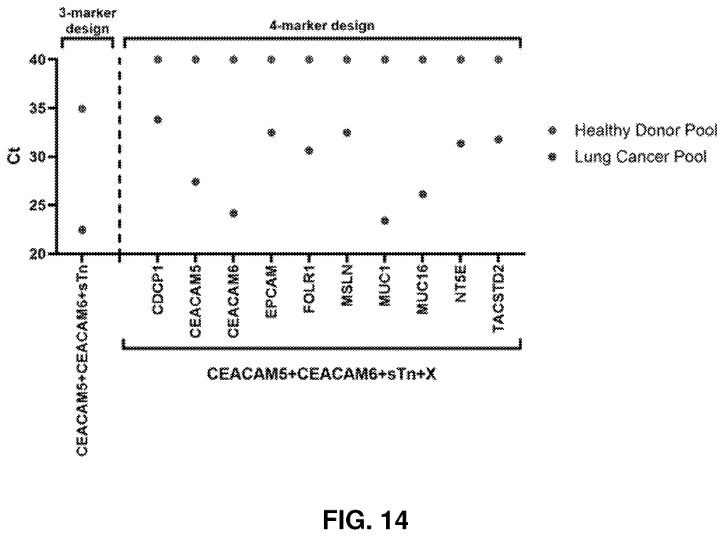

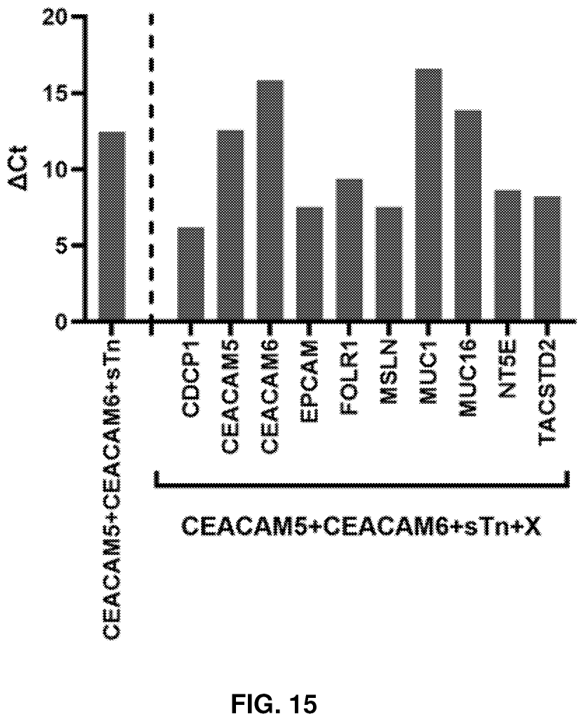

FIGS. 14 and 15 compare the discrimination performance across all combos tested. The 4-biomarker assay design consistently generated distinct signal characteristics than the 3-biomarker design across both sample pools. However, it is observed significantly improved discrimination performance (measured as ΔCt: Cthealthy-Ctcancer) for several 4-biomarker combinations including CEACAM5+CEACAM6+sTn+MUC1 and CEACAM5+CEACAM6+sTn+MUC16, as shown in FIG. 15. This performance improvement was driven by a preferential reduction of healthy background signal relative to cancer signal, resulting in an increase in overall ΔCt. The top performing 4-biomarker combinations exhibited an increase in ΔCt>3, representing a 10-fold increase in signal-to-noise relative to the 3-biomarker assay design. Notably, all 4-biomarker combinations exhibited no detectable signal from the Healthy Donor Pool, demonstrating the high specificity of this approach.

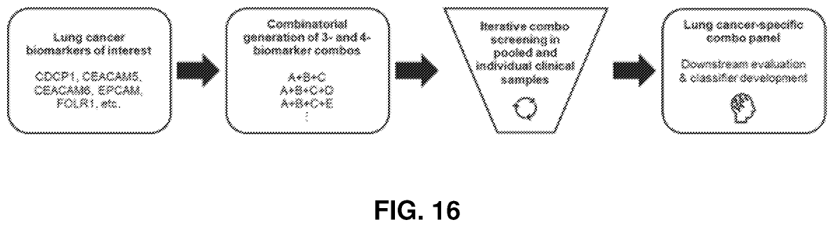

Using the strategy outlined in FIG. 16, screening is performed on hundreds of three- and four-biomarker combinations in clinical samples to identify those with the strongest discrimination performance for LUAD. Briefly, three- and four-biomarker combinations were combinatorially generated based on biomarker candidates with functioning affinity reagents. Combination candidates were screened in multiple rounds of sample sets composed of pooled and individual clinical samples, where the top performing combos are selected for subsequent rounds of testing. This process culminated in the selection of six biomarker combinations to be included in the final assay panel. The final panel included both three-biomarker and four-biomarker combinations.

### Demonstration of Lung Adenocarcinoma Detection in Clinical Samples

The biomarker combination panel is evaluated in 382 individual plasma samples including LUAD cases and non-cancer controls. A summary of the samples included in the present disclosure is provided in Table 1. Each sample was processed via SEC and split equally across all six biomarker combinations. The Ct values generated from each combination were aggregated into a single risk score using an Elastic Net model in a leave-one-out cross-validation (LOOCV). A 90% specificity cutoff was established based on all non-cancer controls (healthy individuals with/without a history of smoking, COPD patients, and subjects with benign tumors, n=290), and the resulting cutoff was used to generate a binary call for each sample. All cases and non-cancer controls were used for model development in LOOCV.

TABLE 1
Demographic information for clinical samples
included in the present disclosure.

| | Healthy controls | COPD | Benign Tumor | LUAD |
|---|---|---|---|---|
| Total subjects | 203 | 42 | 45 | 92 |
| Stage | | | | |
| IA | | | | 44 |
| IB | | | | 24 |
| II | | | | 18 |
| III | | | | 4 |
| IV | | | | 2 |
| History of | | | | |
| smoking | | | | |
| Yes | 101 | 42 | 30 | 64 |
| (current/former) | | | | |
| No | 102 | — | 15 | 26 |
| Unknown | — | — | — | 2 |
| Gender | | | | |
| Male | 82 | 17 | 19 | 47 |
| Female | 121 | 25 | 26 | 45 |
| Age (years) | | | | |
| Median | 59 | 58 | 64 | ND* |
| Mean | 60 | 59 | 62 | ND* |
| <50 | — | — | 8 | — |
| 50-59 | 112 | 26 | 9 | 15 |
| 60-69 | 68 | 13 | 14 | 37 |
| 70-79 | 20 | 3 | 13 | 27 |
| 80-89 | 3 | — | 1 | 13 |
| Ethnicity | | | | |
| White | 165 | 42 | 27 | 27 |
| Black | 28 | — | 1 | 1 |
| Asian | 2 | — | — | — |
| Other | 6 | — | — | — |
| Unknown | 2 | — | 17 | 64 |
| Sample source | | | | |
| Mercy | 203 | — | — | — |
| collection | | | | |
| Ontario | — | — | — | 64 |
| Tumour Bank | | | | |
| ProteoGenex | — | 42 | 9 | 20 |
| iSpecimen | — | — | 36 | 8 |

*Mean and Median were not determined for these samples as only age ranges were provided by the biobanks.

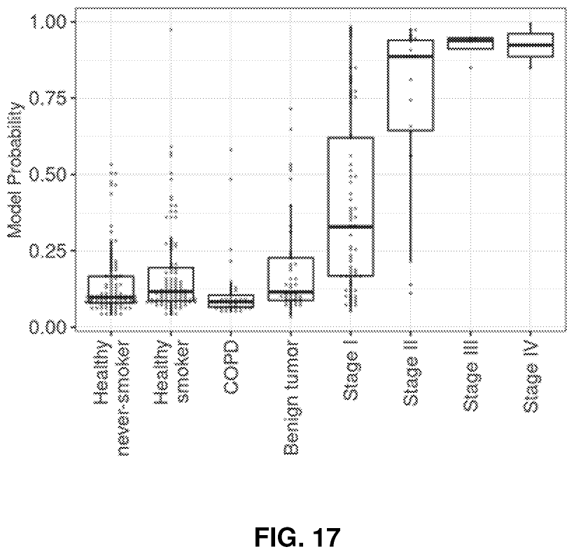

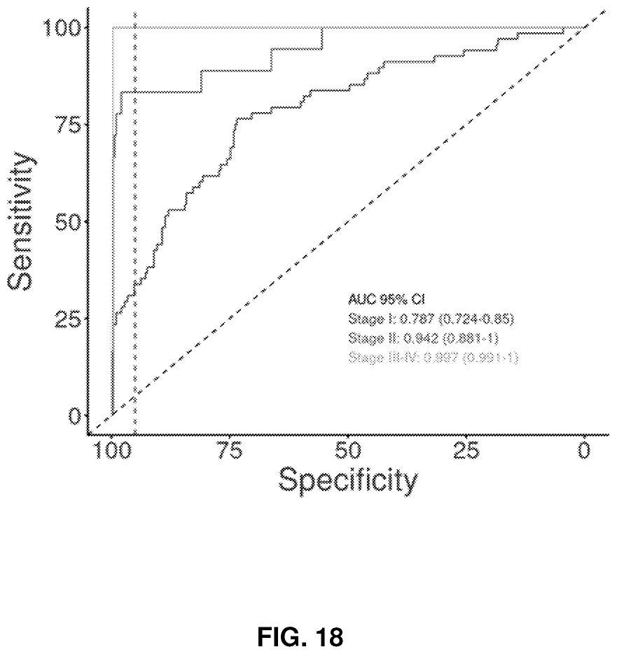

The model scores for each sample group are summarized in FIG. 17. Assay signal was significantly stronger among LUAD cases relative to non-cancer controls and increased with cancer stage. The trained model exhibited an all-stage LUAD sensitivity of 56.5% (95% CI: 43.5%-69.6%) at a specificity of 90% and an AUC of 0.831 (95% CI: 0.780-0.882). The sensitivity and AUC of stage I LUAD were 45.6% (95% CI: 29.4%-60.3%) and 0.787 (95% CI: 0.724-0.850), respectively (FIG. 18). A summary of assay performance across stage and tumor size is provided in Table 2.

TABLE 2
Assay discrimination performance across LUAD
stage and tumor size at 90% specificity.

| | n | Sensitivity (95% CI) | AUC (95% CI) |
|---|---|---|---|
| LUAD Stage | | | |
| Stage I | 31/68 | 45.6% (29.4%-60.3%) | 0.787 (0.724-0.850) |
| Stage II | 15/18 | 83.3% (66.7%-100%) | 0.942 (0.881-1.000) |
| Stage III-IV | 6/6 | 100% (55.2%-100%) | 0.997 (0.991-1.000) |
| All Stage | 52/92 | 56.5% (43.5%-69.6%) | 0.831 (0.780-0.882) |
| Tumor Size* | | | |
| ≤3 cm | 24/59 | 40.7% (23.7%-57.6%) | 0.758 (0.668-0.828) |
| >3 cm | 27/32 | 84.4% (71.8%-93.8%) | 0.960 (0.933-0.987) |

*Tumor size (represented by greatest tumor dimension) available for 91/92 cases.

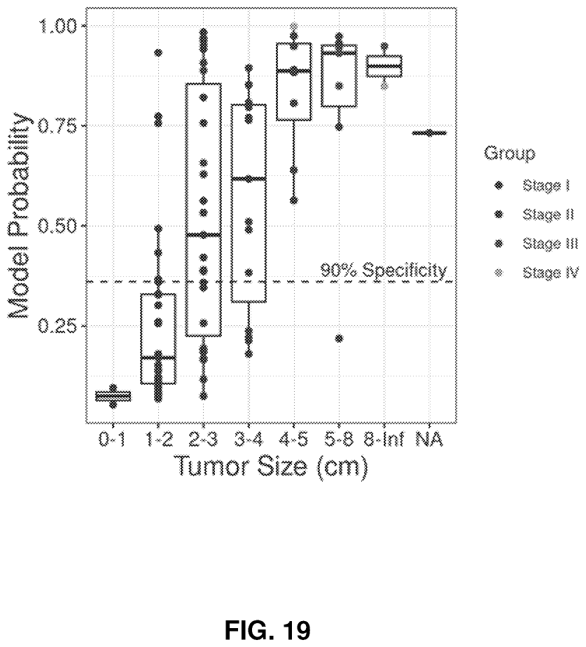

FIG. 19 summarizes assay signal as a function of tumor size and cancer stage. Assay signal was positively correlated with greatest tumor dimension as demonstrated by a Spearman coefficient of 0.66 (p-value=1.5e-12). Signal was also correlated with cancer stage within a given tumor size range, likely due to nodal involvement and/or metastatic disease. The assay (with established cutoff described above) exhibited 40.7% sensitivity among LUAD tumors less than or equal to 3 cm.

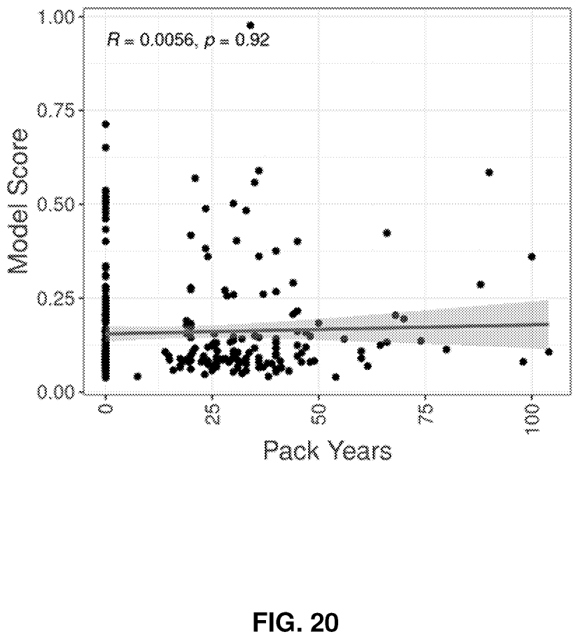

A correlation was not observed between assay signal and tobacco use among cancer-free individuals. Signal distributions between healthy individuals with a smoking history and those without were not statistically different (p-value=0.09922). Moreover, assay signal was not correlated with smoking history (in pack-years) among all non-cancer controls (Spearman coefficient=−0.0043, p-value=0.94) as shown in FIG. 20. Similarly, a correlation was not observed between assay signal and donor sex among healthy individuals with and without a smoking history (Wilcoxon p-value=0.094), while a weak correlation was observed with age (Spearman R=0.14, p-value=0.043).

The present disclosure demonstrates the functionality of a novel immunoassay platform that detects EVPs displaying four distinct colocalized surface biomarkers. The method is used to detect lung adenocarcinoma-derived EVPs and compared the discrimination performance against previously published three-biomarker assay design. Importantly, the data indicate that requiring a fourth colocalized biomarker significantly reduced healthy background signal and improved signal-to-noise for several biomarker combinations. This work culminated in the development of a prototype assay that exhibited discrimination of early-stage LUAD in a case-control study.

The performance of the assay described herein among stage IA LUAD cases was notable, including detection of some tumors less than 2 cm in size. Stage IA tumors represent the majority of LUAD cases detected by LDCT screening, accounting for >55% of prevalent cases and >60% of incident cases during the National Lung Screening Trial. As a result, sensitivity for stage IA LUAD represents a critical performance metric for evaluating emerging blood-based lung cancer tests despite the technical challenge associated with detecting small tumors. While the tumor size regime for stage IA cases (≤3 cm) overlaps with the theoretical detection limit of ctDNA-based methods, the results reported here demonstrate the utility of the EVP-based approach described herein and compare favorably to previously reported lung cancer detection tests.

The strong correlation between assay signal and tumor size suggests the assay described herein is detecting lung cancer-derived EVPs shed into circulation. This is consistent with multiple studies demonstrating that EVP shed rate is positively correlated with tumor size. LUAD cases not detected by the assay were primarily small (>60% missed cases were ≤2 cm) and therefore may represent low concentrations of tumor-derived EVPs in circulation and/or low expression of the target biomarkers. Assay signal was not correlated with tobacco use among cancer-free individuals, suggesting that LUAD-associated EVP populations are not elevated simply due to tobacco exposure. This finding supports the potential application of this technology in both high-risk and average-risk patient populations. Moreover, the discrimination between lung cancer cases and pathologically confirmed benign lung nodules suggests potential utility of this test for lung nodule adjudication.

A key advantage of the immunoassay platform described herein is the attenuation of healthy background signal when multiple cancer-associated biomarkers are colocalized. Nonzero healthy background signal represents a fundamental biological limit for the clinical sensitivity of a biomarker candidate as it obscures the signal derived from an early-stage tumor. This is particularly problematic for bulk biomarker measurements given that cancer-associated markers are often expressed to some degree by one or more healthy tissues. As this baseline expression increases, a tumor must reach a larger size before biomarker concentrations in circulation become elevated.

Several strategies have been proposed to reduce healthy background signal and improve early-stage sensitivity. Longitudinal biomarker measurements establish patient-specific baseline signals which may enable earlier detection of signal increases. However, this strategy is challenging to implement clinically and does not eliminate healthy background interference. Alternative approaches seek to identify highly specific cancer antigens which exhibit extremely low/no expression across healthy tissues, including cancer-specific mutations and novel neoantigens. However, such approaches are limited by tumor biology and may limit detection to very specific patient subpopulations. Conversely, the approach described here leverages known cancer-associated biomarkers which are co-expressed on tumor cells and colocalized on tumor-derived EVPs. The requirement of biomarker colocalization enables selective reduction of background signal from competing tissues and significantly improves signal-to-noise relative to individual biomarker measurements. The improvement in specificity that it is observed using the four-biomarker assay design is consistent with a similar approach reported for prostate cancer EVP detection.

The four-biomarker assay design reported here enables greater selectivity for distinct EVP subpopulations than the previously reported three-marker design. However, this increase in specificity has several limitations which may impact overall discrimination performance. First, four-marker combinations may exhibit significantly lower cancer signal than three-marker combinations, as shown in FIG. 14, resulting in different signal separation between cases and controls. Second, four-biomarker combinations may target more distinct subsets of cancer cases based on their biomarker expression profiles, resulting in fewer detected cases per combination. To address these challenges, signal may be strengthened by increasing the sample input volume (i.e., adding more EVPs to the assay) and/or implementing novel signal amplification strategies. Additionally, sensitivity may be boosted by constructing a panel of complementary biomarker combinations which detect distinct cancer cases.

Assay specificity may be further improved by incorporating additional biomarkers in the assay configuration (e.g., a five-biomarker assay design). This strategy would require an updated DNA design to enable four or more detection probes. However, such an approach may be selected based on the intended application. The optimal assay configuration is ultimately dependent on the specific biomarkers selected and their level of co-expression in cancer/non-cancer tissues.

While the results suggest discrimination of lung adenocarcinoma, Lung cancer plasma samples were collected immediately prior to or at diagnosis, with diagnostic work up triggered by symptomatic presentation, incidental findings, and/or routine screening. As a result, these cases provide relevant information regarding asymptomatic individuals with preclinical disease. In addition, case and control clinical specimens are sourced from multiple vendors and collected within multiple clinical settings which reflect preanalytical factors. This risk will be addressed during future clinical studies which are isolated to patient cohorts collected under a single protocol. Lastly, the present disclosure used a biomarker combination panel focused on LUAD detection which represents a subset of total lung cancer cases. This focus on LUAD was intentional to demonstrate the potential of the technology, and additional assay development may be used to target other lung cancer histologies. Assay sensitivity in other histologies may be addressed by building additional biomarker combinations which target those distinct biomarker expression profiles.

The present disclosure provides an EVP-based technology for the detection of early-stage lung adenocarcinoma. The biomarker combination panel and associated classifier support use of the EVP assay as a blood-based strategy for early lung cancer detection.

### INCORPORATION BY REFERENCE

References and citations to other documents, such as patents, patent applications, patent publications, journals, books, papers, web contents, publicly accessible databases, have been made throughout this disclosure. All such documents are hereby incorporated herein by reference in their entirety for all purposes.

### EQUIVALENTS

Various modifications of the invention and many further embodiments thereof, in addition to those shown and described herein, will become apparent to those skilled in the art from the full contents of this document, including references to the scientific and patent literature cited herein. The subject matter herein contains important information, exemplification and guidance that can be adapted to the practice of this invention in its various embodiments and equivalents thereof.

1. A method of detecting a condition in a subject, the method comprising: (a) capturing a particle from a biological sample obtained from the subject with a capture agent that binds a first biomarker associated with the particle; and (b) detecting co-localized second, third, and fourth biomarkers on the captured particle to determine the presence of the condition.

2. The method of claim 1, wherein the biological sample is selected from the group consisting of whole blood, plasma, serum, sputum, urine, saliva, cerebrospinal fluid, ascites fluid, pleural effusion, and combinations thereof.

3. The method of claim 1, wherein the particle is an extracellular vesicle selected from the group consisting of an exosome, a microvesicle, an apoptotic body, and an oncosome.

4. The method of claim 1, wherein the particle is a non-vesicular particle selected from the group consisting of an exomere, a supermere, a lipoprotein particle, a proteomic aggregate, and a genomic aggregate.

5. The method of claim 1, wherein the first biomarker is associated with the condition and comprises a protein selected from EGFR and CEACAM5, or a carbohydrate selected from SialylTn and Sialyl Lewis X.

6. The method of claim 1, wherein the first biomarker is not specific to the condition and is a pan-extracellular vesicle biomarker selected from the group consisting of CD9, CD63, CD81, TSPAN1, TSPAN2, TSPAN3, an ITGA family integrin, an ITGB family integrin, and combinations thereof.

7. The method of claim 1, wherein the capture agent comprises a lectin or other glycan-binding affinity reagent that binds a carbohydrate or glycan structure broadly present on extracellular vesicles.

8. The method of claim 1, wherein the capture agent binds a lipid component of the particle selected from phosphatidylserine and a glycolipid.

9. The method of claim 1, wherein the capture agent captures particles based on electrostatic interactions and comprises an ion-exchange material or a charged surface.

10. The method of claim 1, wherein diagnostic specificity is conferred by the detection of the co-localized second, third, and fourth biomarkers rather than by the capture agent.

11. The method of claim 1, wherein the second, third, and fourth biomarkers are each independently selected from the group consisting of a protein, a carbohydrate, a lipid, and a nucleic acid.

12. The method of claim 1, wherein at least one of the co-localized second, third, and fourth biomarkers comprises a different epitope of the first biomarker than the epitope bound by the capture agent.

13. The method of claim 1, further comprising detecting a fifth co-localized biomarker, a sixth co-localized biomarker, or both, on the captured particle.

14. The method of claim 1, wherein detecting the co-localized biomarkers comprises contacting the captured particle with a plurality of probes, each probe comprising (i) a binder specific to one of the co-localized biomarkers and (ii) an oligonucleotide linked to the binder, wherein the oligonucleotides are configured to form an amplifiable nucleic acid product by ligation only when the probes are bound to biomarkers co-localized on the same particle.

15. The method of claim 14, wherein detecting further comprises amplifying the amplifiable nucleic acid product by qPCR or dPCR, or detecting the amplifiable nucleic acid product by next-generation sequencing.

16. The method of claim 1, wherein the condition is a cancer selected from the group consisting of lung cancer, ovarian cancer, pancreatic cancer, breast cancer, and colorectal cancer.

17. The method of claim 1, further comprising: measuring a multi-marker co-localization signal in each of a plurality of samples taken from the subject over time; calculating an elimination rate from at least two of the multi-marker co-localization signals; and determining a treatment efficacy based on the elimination rate.

18. A kit for detecting a condition, the kit comprising: (a) a capture agent configured to bind a first biomarker associated with a particle; (b) a plurality of detection probes configured to bind co-localized second, third, and fourth biomarkers on the captured particle, each detection probe comprising a binder and an oligonucleotide linked thereto; (c) reagents for performing a proximity-dependent ligation reaction to form an amplifiable nucleic acid product; and (d) instructions for use.

19. The kit of claim 18, wherein the capture agent is configured for pan-extracellular vesicle capture and comprises an affinity reagent specific for a tetraspanin, an integrin, a carbohydrate or glycan broadly present on extracellular vesicles, a membrane lipid, or a charged surface feature.

20. A system for detecting a condition, the system comprising: (a) a capture substrate comprising immobilized capture agents configured to bind a first biomarker associated with a particle; (b) a plurality of detection probes configured to bind co-localized second, third, and fourth biomarkers on the captured particle; and (c) an instrument configured to generate a quantitative readout indicative of particles displaying a multi-marker co-localization signature.
